(19) United States
(12) Patent Application Publication
(10) Pub. No.: US 2026/0185163 A1
(43) Pub. Date: Jul. 2, 2026

(54) MEN1 MUTATIONS AND USES THEREOF

(71) Applicants: DANA-FARBER CANCER INSTITUTE, INC., Boston, MA (US); MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Scott A. Armstrong, Wayland, MA (US); Florian Perner, Boston, MA (US); Ross L. Levine, New York, NY (US); Eytan M. Stein, New York, NY (US); Sheng F. Cai, New York, NY (US)

(21) Appl. No.: 19/132,214

(22) PCT Filed: Nov. 21, 2023
(86) PCT No.: PCT/US2023/080796

§ 371 Date: May 22, 2025

Related U.S. Application Data
(60) Provisional application No. 63/384,722, filed on Nov. 22, 2022.

(51) Int. Cl. C12Q 1/6886

(52) U.S. Cl. CPC C12Q 1/6886; C12Q 2600/106; C12Q 2600/156

(57) ABSTRACT

Disclosed are mutations in the MEN1 gene of cancer cells that affect sensitivity of the cells to menin-inhibitory therapeutics. Also disclosed are diagnostic tests to detect the mutations. Diagnostic testing of cancer cells from a patient undergoing therapy with menin inhibitors can indicate that the therapy should be changed or stopped. Diagnostic of cells from a patient prior to treatment with a menin inhibitors can indicate an alternative therapy should be used.

This application claims priority to U.S. Provisional Application No. 63/384,722, filed Nov. 22, 2022, the entire contents of which are incorporated herein by reference.

### GOVERNMENT INTERESTS

This invention was made with government support under Grant No. CA176745, CA206963, CA204639, CA066996 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to methods for diagnosing resistance to a menin-inhibitory therapeutic and detecting MEN1 mutations in a subject having cancer or suspected of having cancer.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND OF THE INVENTION

Menin is a chromatin adaptor protein involved in formation and stability of conserved multiprotein complexes on chromatin, including Mixed Lineage Leukemia 1 (MLL1: KMT2A) and MLL2 (KMT2B) histone methyltransferase complexes and the AP-1 transcription factor complex.

### SUMMARY OF THE INVENTION

Resistance to menin inhibitors can arise in cancer patients receiving these drugs. Disclosed here are mutations in MEN1 that give rise to resistance to multiple classes of menin inhibitors. Disclosed are methods for diagnosing resistance to menin-inhibitory therapeutics in a subject having cancer or suspected of having cancer. Disclosed are methods for diagnosing resistance of a cancer to a menin inhibitor. Disclosed are methods for treating a cancer patient who is receiving a menin-inhibitory therapeutic.

Disclosed are methods for diagnosing resistance to menin-inhibitory therapeutics in a subject having cancer or suspected of having cancer by detecting a mutation in a MEN1 gene in the subject. The mutation can be detected in a cell from the cancer. The cancer can be dependent on menin protein. The cancer can be an acute leukemia. The acute leukemia can be a myeloid or lymphoblastic acute leukemia. In some embodiments, the cells of the cancer can have a rearrangement of a Mixed Lineage Leukemia gene (MLLr) (also called KMT2A) or a Nucleophosmin gene (NPM1c). The protein encoded by the MMLr contributes to a malignant phenotype of the cancer in presence of menin. The menin-inhibitory therapeutics can block or decrease interaction of menin protein with a MLL1/MLLr protein and/or decrease drug-induced displacement of a menin complex from chromatin. The menin-inhibitory therapeutics can be of any type. The subject can have been treated with a menin-inhibitory therapeutic. The subject can be relapsed or no longer responsive to menin-inhibitory therapeutics.

The mutation that is detected using the disclosed methods can substitute an amino acid for a wild-type amino acid in menin protein at amino acids 327, 331, 349, 160, or combinations thereof. The wild-type amino acid at position 327 can be methionine, at position 331 glycine, at position 349 threonine, at 160 serine, or combinations thereof. The wild-type amino acid can be substituted with an isoleucine, valine, arginine, aspartic acid, methionine, cysteine or combinations thereof. The wild-type amino acid can be substituted with an isoleucine or valine at position 327, an arginine or aspartic acid at position 331, a methionine at position 349 and/or a cysteine at position 160. Multiple of the mutations can be present.

Disclosed are methods for diagnosing resistance of a cancer to a menin inhibitor. A cell sample can be obtained from the cancer. The cells can be tested for presence of a mutation in the MEN1 gene.

Disclosed are methods for treating a cancer patient who is receiving a menin-inhibitory therapeutic. A genome from a cell from the cancer can be tested for presence of a MEN1 mutation. If a MEN1 mutation is detected, treatment of the cancer patient with the menin-inhibitory therapeutic can be changed. The treatment can be discontinued.

Other objects and advantages of this invention will become readily apparent from the ensuing description.

### BRIEF DESCRIPTION OF THE FIGURES

FIG. 1 is a representation showing Menin-inhibitor resistance is associated with emergence of MEN1 mutations. Panel A is a graphical depiction of the percentage (%) of leukemic blasts in the peripheral blood of example patients during SNDX-5613 treatment in the AUGMENT-101 phase-1 clinical trial. Clinical events are marked with arrows and labeled respectively. Panel B is a schematic showing the fraction of patients in which MEN1-M327I, -M327V, -G331R, -G331D or -T349M was detected by droplet digital PCR (ddPCR). For this analysis we included patients that received at least two cycles of SNDX-5613 treatment (>56 days) and had 2 or more mutant droplets detected. Panel C provides pie charts displaying the fraction of MEN1-mutant alleles measured by ddPCR at the time point of screening and relapse in 4 individual patients from the cohort shown in Panel B. Panel D provides longitudinal kinetics of MEN1-mutant selection in two patients from the cohort shown in Panel B. Mutant allele frequencies at different timepoints during SNDX-5613 treatment were analyzed by ddPCR. Panels E, F, and G provide graphical displays of examples of the percentage (%) of human leukemia cells in the peripheral blood of individual NOG-mice during a long-term patient-derived xenograft (PDX) treatment trial with the Menin-inhibitor VTP-50469 (0.03% rodent diet). Blue bars in the background mark the time of oral Menin inhibitor exposure via drug-supplemented rodent diet. MEN1 mutations detected via targeted DNA-sequencing in individual animals of Panel E) PDX 1 (MLL::AF6), Panel F) PDX 2 (NPM1c) or Panel G) PDX 3 (MLL::AF10) are labeled and marked with arrows.

FIG. 2 is a representation showing base-editor screening identifies recurrent MEN1 mutations mapping to the MLL1-binding pocket. Panel A provides a dot-plot showing example results of a CRISPR-Cas9 base-editor screen in MOLM13 cells aiming to identify point mutations that cause resistance to Menin inhibitor treatment. Each dot represents a single guide RNA. Along the x-axis, guide RNAs are sorted by their targeting location relative to the Menin-coding sequence. The y-axis shows differential CRISPR-beta-scores (DMSO-score subtracted from the VTP-50469-treatment score). Outstanding hits are marked in red and targeted amino acid residues are labeled. Panel B provides an X-ray co-crystal structure of SNDX-5613 bound to WT-Menin (PDB: 7UJ4). The hydrogen bonds between sulfonamide oxygen of SNDX-5613 and indole nitrogen of W346 or sulfonamide nitrogen of SNDX-5613 and backbone carbonyl oxygen of M327 are indicated with black dashed lines. Nonpolar hydrogens are shown for SNDX-5613 and the W346. Panel C provides an X-ray co-crystal structure of Menin in complex with MLL14-15 peptide (PDB: 4GQ6). View corresponds to FIG. 7D. Recurrently mutated amino acids are labeled in red. The W346 residue that builds up a strong hydrogen bond with SNDX-5613 to stabilize binding of the molecule is marked in blue. Panel D is a representation showing the structure alignment between co-crystal structure of SNDX-5613 bound to M327I-mutant Menin (PDB: 8E90) and SNDX-5613 bound to WT Menin (PDB: 7UJ4). SNDX-5613 is colored in yellow in WT Menin, and magenta in M327 mutant co-crystal structure. The magenta dashed lines indicate large distances, incapable of H-bond interactions, between SNDX-5613 in the M327I-mutant and the WT Menin protein and are in contrast to H-bond between W346 and SNDX-5613 in the WT Menin highlighted in black dashed line as in Panel B. Panel E provides the fluorescence polarization assay measuring dose-dependent displacement of an MLL1 peptide from WT, M327I- and T349M-mutant Menin under treatment with SNDX-5613, MI-3454 or DS-25, a compound from the Daiichi-Sankyo Menin-inhibitor series. Panel F provides the fluorescence polarization assay probing the binding affinity of a MLL1 peptide to WT. M327I- and T349M-mutant Menin.

FIG. 3 provides is a representation showing example MEN1 mutations confer resistance to Menin-inhibitor treatment in vitro. Panel A and Panel B provides example dose-response curves of Panel A) MOLM13 (MLL::AF9) and Panel B) OCI-AML3 (NPM1c) cells to SNDX-5613 upon expression of MEN-M327I, -G331R, -T349M or -WT. Cell counts were measured by flow cytometry and displayed relative to the DMSO control (mean+/−SEM, n=4, each 3 technical replicates). Panel C provides is a schematic depicting the CRISPR-Cas9 gene editing strategy utilized to insert the M327I mutation into the endogenous MEN1-locus. Panel D provides example growth curves showing expansion of MV4;11 (MLL::AF4) and OCI-AML3 (NPM1c) bulk cell populations overtime after nucleofection under treatment with 50 nM SNDX-5613 (mean+/−SD, n=3). Panel E provides curves depicting example fractions of the MEN1-M327I mutant allele detected by ddPCR in MV4;11 and OCI-AML3 bulk cell populations over time under treatment with 50 nM SNDX-5613 (in vitro clonal selection assay) (mean, n=1, DNA pooled from 3 replicates). Panel F and Panel G provide dose-response curves showing example sensitivities of MV4;11 (MLL::AF4) cells harboring MEN1-M327I mutations to Panel F) SNDX-5613. Panel G) MI-3454 and the Daiichi-Sankyo compound. Panel H provides example dose-response curves showing the sensitivity of MV4;11 (MLL::AF4) cells harboring MEN1-T349M mutations to SNDX-5613, MI-3454 and the Daiichi-Sankyo compound. Panel F, Panel G and Panel H cell counts were measured by flow cytometry and displayed relative to the DMSO control (mean+/−SEM, n=4, each 3 technical replicates).

FIG. 4 provides is a representation showing Menin chromatin binding and aberrant gene expression is rescued by example MEN1 mutations. Panel A provides torpedo-plots of total Menin signal intensity around transcription start sites (TSS) from ChIP-sequencing (ChIPseq) in MV4;11-MEN1-WT and -M327I mutant cells treated with SNDX5613 (0.1p M, 1 μM, 5 μM) or DMSO as control. Panel B provides is a graph showing read-normalized Menin-TSS-signal at MLL1-target genes in MV4;11 cells under SNDX5613 treatment (mean+/−SD, 3000 data points per condition). Panel C provides example ChIPseq tracks of Menin and MLL1 at the MEIS1-locus in MV4;11 cells under SNDX5613 treatment (representative example of 3 replicates). Panel D provides torpedo-plots of Menin signal intensity around transcription TSS from ChIPseq in MEN1-WT and -T349M mutant PDX3 treated with VTP-50469 for 14 days. Panel E provides is a graph showing example Menin-TSS-signal at MLL1-target genes in PDX3 (mean+/−SD, 3000 TSS data points per condition). Panel F provides is a graph showing example read-normalized MLL1-TSS-signal at sites that lose >80% of Menin in WT cells treated with VTP-50469 (mean+/−SD, 293 data points per condition). Panel G provides example ChIPseq tracks of Menin and MLL1 at the MEIS1-locus and HOXA-cluster in PDX3. Panel H and Panel I provide heatmaps of example RNAseq data showing the expression dynamics of all genes that are differentially expressed (DEGs) under treatment with a Menin-inhibitor in MV4 (Panel H); 11 cells or PDX3 (Panel I). Kmeans clustering (4) was applied to generate heatmaps, representative genes are used for annotation.

FIG. 5 provides is a representation showing example new MEN1 mutations detected in patients upon relapse on SNDX-5613. Panel A. Panel B, and Panel C provides tables showing example results of the IMPACT targeted DNA-sequencing panel from patient 1-4 at the time point of screening prior to enrolling on the AUGMENT-101 trial and at relapse on SNDX-5613 treatment. MEN1 mutations are highlighted in red. Panel D provides pie charts displaying example fractions of MEN1-mutant alleles measured by droplet digital PCR (ddPCR) at the time point of relapse (or last available sampling time point before relapse) in all individual patients from the cohort shown in FIG. 1B. Relative mutation frequencies (number of MEN1-mutant/WT droplets) are labeled in white. Mutations which were detected in >2 droplets were considered. Panel E provides longitudinal kinetics of MEN1 mutant selection in two selected patients from the cohort shown in FIG. 1B. Mutant allele frequencies at different time points during SNDX-5613 treatment were analyzed by ddPCR.

FIG. 6 provides a representation showing the development of Menin-inhibitor resistance in a KMT2Ar PDX. Panel A provides a box-plot (mean+/−SD) showing the percentage (%) of human leukemia cells in the bone marrow of NOG-mice transplanted with PDX3 at baseline (n=4), 4 weeks (w) (n=4), 8 w (n=5) and at 10-12 w (n=5; symptomatic leukemia relapse) on Menin-inhibitor treatment. Dots represent individual animals. Panel B provides box-plots (mean+/−SD) showing the mean fluorescence intensity (MFI) of the myeloid differentiation markers CD11b, CD13 and CD14 on the cell surface of human cells detected in the bone marrow of NOG-mice transplanted with PDX3 at baseline (n=4), 4 w (n=5), 8 w (n=4) and at 10-12 w (n=4) on Menin-inhibitor treatment. Dots represent individual animals. Panel C provides bone marrow cytology pictures (cytospins) from each of two representative animals at baseline, 4 w, 8 w and 12 w on Menin-inhibitor treatment. Panel D provides pie charts showing the fraction of MEN1-T349M (red) as compared to MEN1-WT (blue) measured by droplet digital PCR (ddPCR) at baseline, 8 weeks and 12 weeks (fulminant clinical relapse) in human cells isolated from PDX3 mice and purified using magnetic cell sorting.

FIG. 7 provides a representation showing base-editor screening as a tool to identify point mutants in MEN1. Panel A provides a schematic depicting an example workflow of the MEN1-base editor screen performed in MOLM13 (MLL::AF9) and MV4;11 (MLL::AF4) cells. Panel B provides a dot-plot showing example results of a CRISPR-Cas9 base-editor screen in MV4;11 cells aiming to identify point mutations that cause resistance to Menin inhibitor treatment. Each dot represents a single guide RNA. Along the x-axis guide RNAs are sorted by their targeting location relative to the Menin-coding sequence. The y-axis shows differential CRISPR-beta-scores (DMSO-score subtracted from the VTP-50469-treatment score). Outstanding hits are marked in red and targeted amino acid residues are labeled. Panel C provides a representation showing the alignment of the Menin bound SNDX-5613 (PDB: 7UJ4) with Menin bound MLL14-15 peptide (PDB: 4GQ6). Recurrently mutated amino acids are labeled in red. The W346 residue that builds up a strong hydrogen bond with SNDX-5613 to stabilize binding of the molecule is marked in blue.

FIG. 8 provides a representation showing atomic modeling of Menin and its mutations using equilibrium simulations. Panel A provides example trajectory length distributions for equilibrium simulations of Menin wild-type and mutants (rows). Simulations were all run simultaneously on Folding@home and the frequency distribution of their simulation lengths is indicated for each construct, both with and without SNDX-5613 (left and right columns, respectively. Panel B provides example equilibrium molecular dynamics simulations and Markov models reveal that helices contacting SNDX-5613 separate upon mutation. Distance distributions between the sulfonamide contacting helices were computed for WT Menin and each mutant. Panel C provides DiffNets analysis comparing WT to mutant Menin using backbone features showing helical separation (blue lines) around SNDX-5613 (magenta). Dashed lines indicate helical motion as a structural feature that significantly differs between WT and mutant Menin. Blue lines indicate that helices move further apart and separate upon mutation, while red dashed lines indicate that helices come closer together. Panel D provides example timescales after clustering all Folding@home simulations. Based on this plot, a lag time of 7 nanoseconds was chosen for MSM construction to ensure Markovanaity.

FIG. 9 provides a representation showing how example MEN1 mutations impact binding affinity of SNDX-5613 to the MLL1/2 binding pocket. Panel A provides example titration curves of WT-, M327I- and T349M-mutant Menin against a FITC-conjugated MLL1 4-43(C-A) peptide probe (n=3) for determination of equilibrium dissociation constant (Kd). Panel B provides example curves depicting the fraction of SNDX-5613 (left) or MLL1 (right) bound to Menin (WT or mutant) over time determining the molecule's dissociation rates (off-rates) over time. Panel C provides example isothermal titration calorimetry assay measuring the binding of SNDX-5613 to WT-, M327I and T349M-mutant Menin confirming the mutation inflicted shift in affinity detected using the fluorescence polarization assay (FIG. 2e). Panel D provides example titration curves of WT-, M327I- and T349M-mutant Menin against a FITC-conjugated MLL2 (15-48) peptide probe (n=3). Panel E provides fluorescence polarization assay measuring example dose-dependent displacements of an MLL2 peptide from WT, M327I- and T349M-mutant Menin under treatment with SNDX-5613 or MI-3454.

FIG. 10 provides a representation showing lentiviral expression of MEN1 mutants confers resistance in cell lines. Panel A provides an example western blot in MOLM13 cells showing expression of HA-tagged MEN1-WT and M327I-mutant construct. Panel B provides example dose-response curves of MOLM13 cells to SNDX-5613 upon expression of MEN1 mutants compared to -WT. Cell counts were measured by flow cytometry and displayed relative to the DMSO control (mean+/−SEM, n=4, each 3 technical replicates). Panel C provides example dose-response curves of MV4;11 and OCI-AML3 cells to SNDX-5613 upon expression of MEN1 mutants or -WT measured by Cell-titerGlo (mean+/−SD, n=3). Panel D and Panel E provide representations showing example induction of differentiation by SNDX-5613 in OCI-AML3 cells expressing MEN1 mutants compared to WT measured by Panel D) flow cytometry (MFI CD11b) (n=3) and Panel E) cyto-morphology assessment by a hematopathologist (n=3). Panel F provides dose-response curves of OCI-AML3 cells to SNDX-5613 (top panel) or MI-3454 (bottom panel) upon expression of MEN1 mutants compared to -WT. Cell counts were measured by flow cytometry and displayed relative to the DMSO (mean+/−SEM, n=4, each 3 technical replicates). Panel G provides an example Western blot in MV4;11 cells showing expression of HA-tagged MEN1-WT and -mutant constructs. Panel H provides example dose-response curves of MV4;11 cells to SNDX-5613 upon expression of MEN1 mutants compared to -WT. Cell counts were measured by flow cytometry and displayed relative to the DMSO (mean+/−SEM, n=4, each 3 technical replicates).

FIG. 11 provides a representation showing MEN1-M327I endogenous gene-editing induces drug resistance to different Menin-inhibitors in leukemia cell lines. Panel A provides example Sanger-sequencing tracks showing gene-editing in MV4;11 and OCI-AML3 cells generating stable cell lines harboring the mutations indicated above the respective plots at the endogenous MEN1-locus. Panel B provides example dose-response curves of M327I homozygous or -WT MV4;11 cells to a high-dose range of SNDX-5613. Panel C provides example dose-response curves of M327I heterozygous or -WT MV4;11 cells to MI-3454. Panel D provides example dose-response curves showing the sensitivity of OCI-AML3 (NPM1c) cells harboring the MEN1-M327I mutation to SNDX-5613, MI-3454 and the Daiichi-Sankyo compound. Panel E provides example dose-response curves showing the sensitivity of OCI-AML3 (NPM1c) cells harboring the MEN1-T349M mutation to SNDX-5613, MI-3454 and the Daiichi-Sankyo compound. Panel F provides example dose-response curves showing the sensitivity of MV4; II cells harboring homozygous or heterozygous MEN1-M327I mutations to the covalent binder MI-89. Panel G provides example dose-response curves of S160C or -WT MV4;11 cells to SNDX-5613. Panels B-G) Cell counts were measured by flow cytometry and displayed relative to the DMSO control (mean+/−SEM, n=4, each 3 technical replicates). Panel H provides example fluorescence-based cell competition assays measuring relative cell fitness of MV4;11-MEN1-WT, -M327I or -T349M mutant cells in the presence or absence of SNDX-5613 (100 nM) over the course of 21 days by flow cytometry.

FIG. 12 provides a representation showing ChIPseq of Menin and MLL1 in MEN1-WT and -M327I-mutant cells. Panel A provides example ChIPseq tracks of Menin and MLL1 at the PBX3, MEF2C, JMJD1C-loci and the HOXA-cluster in MV4;11 cells under SNDX-5613 treatment (representative example of 3 replicates). Panel B provides example bar graphs showing Menin-ChIP-qPCR results at the MEIS1, MEF2C and HOXA10 transcription start sites after treatment with 100 nM SNDX-5613 or DMSO as control (4 days treatment).

FIG. 13 provides a representation showing MEN1 mutations abrogate changes in gene expression signatures in MV4;11 cells upon SNDX-5613 treatment. Panel A provides example Geneset-enrichment analysis (GSEA) from SNDX-5613 (100 nM, 1 μM or 5 μM) vs. DMSO treated MV4;11 cells harboring the MEN1-M327I mutation or -WT as control. Plotted are the False-discovery rate (FDR) q-values (y-axis) over the normalized enrichment scores (x-axis). Each dot represents a gene set. Relevant genesets covering MLL/HOX-related or myeloid differentiation associated terms were chosen for the analysis and selected terms are annotated. Panel B provides example GSEA plots from SNDX-5613 (100 nM, 1 μM or 5 μM) vs. DMSO treated MV4;11 cells harboring the MEN1-M327I mutation or -WT as control. GSEA was performed for MLL-fusion targets (Olsen et al., Mol. Cell, 2022) and the BROWN_MYELOID_CELL_CEVELOPMENT_UP geneset. Normalized enrichment scores and FDR q-values are indicated below each plot.

FIG. 14 provides a representation showing MEN1 mutations blunt repression of key MLL-target genes upon SNDX-5613 treatment. Panel A provides bar graphs showing example relative gene expression of MEIS1 and MEF2C in MEN1-M327I homozygous (left) and heterozygous (right) cells under treatment with a wide range of SNDX-5613 doses (mean+/−SD, n=3, each measured in triplicates) measured by quantitative real-time PCR using pre-validated Tagman® probes. Panel B provides bar graphs showing example relative gene expression of MEIS1 (left) and MEF2C (right) in MEN1-M327I and MEN1-T349M mutant cells under treatment with a wide range of SNDX-5613 doses (mean+/−SD, n=3, each measured in triplicates) measured by quantitative real-time PCR using pre-validated Taqman® probes. Panel C provides bar graphs showing relative gene expression of MEIS1, PBX3 and HOXA7 in MEN1-T349M-mutant or WT PDX2 treated for 12 days with VTP-50469 (mean+/−SD, n=3, each measured in triplicates, replicates represent individual mice) measured by quantitative real-time PCR using pre-validated Tagman® probes.

FIG. 15 provides a graph showing example longitudinal kinetics of MEN1-mutant selection as assessed by droplet digital PCR in a patient enrolled on the AUGMENT-101 study and treated with SNDX-5613. The y-axis depicts the ratio of mutant:wild-type droplets detected at each time point.

FIG. 16 provides a schematic showing the structural alignment between the co-crystal structure of SNDX-5613 bound to M327I-mutant Menin and SNDX-5613 bound to wild-type Menin. SNDX-5613 is colored in yellow in wild-type Menin, and Magenta in the M327I-mutant structure. The magenta dashed lines indicate large distances, incapable of forming H-bond interactions, between SNDX-5613 in the M327I-mutant. In contrast, for wild-type Menin, there is an H-bond established between residue W346 and SNDX-5613, highlighted via black dashed line.

### DETAILED DESCRIPTION OF THE INVENTION

Menin has been demonstrated to be involved in development and maintenance of certain cancers, including acute leukemias driven by rearrangements involving MLL1 (MLL or KMT2A) or truncating mutations of the Nucleophosmin gene (NPM1c). To leverage this, a series of small molecule inhibitors that disrupt the Menin-MLL1 protein-protein interaction have been developed. Due to their potent activity in pre-clinical models, including the ability to eradicate disease, several of these menin-inhibitors recently entered phase 1 clinical trials (NCT04065399, NCT04067336. NCT04811560, NCT04988555. NCT04752163, NCT05153330). SNDX-5613, currently an advanced clinical compound, has been reported to be safe and efficacious in patients with relapsed or refractory acute leukemia.

This application discloses mutations in the MEN1 gene that make cells resistant to menin-inhibitory drugs. Also disclosed are methods for diagnosing resistance to a menin-inhibitory therapeutic in a subject having cancer or suspected of having cancer, comprising detecting a mutation in a MEN1 gene in a subject. In some embodiments, the cancer comprises acute myeloid leukemia (AML). In some embodiments, the subject has been treated with a menin-inhibitory therapeutic, such as SNDX-5613.

Detailed descriptions of one or more preferred embodiments are provided herein. However, the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Acquired MEN1 Mutations and Resistance to Menin-Inhibitory Therapeutics

In some embodiments, this application relates to cancer cells that have a rearrangement of a Mixed Lineage Leukemia gene (MMLr) (also called KMT2A) or a Nucleophosmin gene (NPM1c). In some embodiments, menin protein contributes to these cancers. In some embodiments, these cancers are dependent on menin protein.

Menin is a chromatin adaptor protein involved in the formation and stability of highly conserved multiprotein complexes on chromatin, including Mixed Lineage Leukemia 1 (MLL1; KMT2A) and MLL2 (KMT2B) histone methyltransferase complexes and the JUND transcription factor complex. Menin is involved in the development and maintenance of certain cancers, including acute leukemias driven by rearrangements involving MLL1 (KMVT2Ar) or truncating mutations of the Nucleophosmin gene (NPM1c). Menin can be involved in other cancers. The protein menin is encoded by the MEN1 gene, which is mutated in patients with multiple endocrine neoplasia type 1 (MEN1) syndrome. Although menin acts as a tumor suppressor in endocrine organs, it participates in leukemic transformation in mouse models. While not wishing to be bound by theory, Menin may possess these dichotomous functions because it interacts with a multitude of proteins with diverse functions in different cellular backgrounds. Herein are disclosed methods for identifying resistance to a menin-inhibitory therapeutic, comprising detecting a mutation in a MEN1 gene.

Tables 1, 2 and 3 illustrate nucleic acid sequences encoding menin and an amino acid sequence of the menin protein.

TABLE 1
Nucleic Acid Sequence of Open Reading
Frame Encoding Menin

ATGGGGCTGAAGGCCGCCCAGAAGACGCTGTTCCCGCTGCGCTCC
ATCGACGACGTGGTGCGCCTGTTTGCTGCCGAGCTGGGCCGAGAG
GAGCCGGACCTGGTGCTCCTTTCCTTGGTGCTGGGCTTCGTGGAG
CATTTTCTGGCTGTCAACCGCGTCATCCCTACCAACGTTCCCGAG
CTCACCTTCCAGCCCAGCCCCGCCCCCGACCCGCCTGGCGGCCTC
ACCTACTTTCCCGTGGCCGACCTGTCTATCATCGCCGCCCTCTAT
GCCCGCTTCACCGCCCAGATCCGAGGCGCCGTCGACCTGTCCCTC
TATCCTCGAGAAGGGGGTGTCTCCAGCCGTGAGCTGGTGAAGAAG
GTCTCCGATGTCATATGGAACAGCCTCAGCCGCTCCTACTTCAAG
GATCGGGCCCACATCCAGTCCCTCTTCAGCTTCATCACAGGTTGG
AGCCCAGTAGGCACCAAATTGGACAGCTCCGGTGTGGCCTTTGCT
GTGGTTGGGGCCTGCCAGGCCCTGGGTCTCCGGGATGTCCACCTC
GCCCTGTCTGAGGATCATGCCTGGGTAGTGTTTGGGCCCAATGGG
GAGCAGACAGCTGAGGTCACCTGGCACGGCAAGGGCAACGAGGAC
CGCAGGGGCCAGACAGTCAATGCCGGTGTGGCTGAGCGGAGCTGG
CTGTACCTGAAAGGATCATACATGCGCTGTGACCGCAAGATGGAG
GTGGCGTTCATGGTGTGTGCCATCAACCCTTCCATTGACCTGCAC
ACCGACTCGCTGGAGCTTCTGCAGCTGCAGCAGAAGCTGCTCTGG
CTGCTCTATGACCTGGGACATCTGGAAAGGTACCCCATGGCCTTA
GGGAACCTGGCAGATCTAGAGGAGCTGGAGCCCACCCCTGGCCGG
CCAGACCCACTCACCCTCTACCACAAGGGCATTGCCTCAGCCAAG
ACCTACTATCGGGATGAACACATCTACCCCTACATGTACCTGGCT
GGCTACCACTGTCGCAACCGCAATGTGCGGGAAGCCCTGCAGGCC
TGGGCGGACACGGCCACTGTCATCCAGGACTACAACTACTGCCGG
GAAGACGAGGAGATCTACAAGGAGTTCTTTGAAGTAGCCAATGAT
GTCATCCCCAACCTGCTGAAGGAGGCAGCCAGCTTGCTGGAGGCG
GGCGAGGAGCGGCCGGGGGAGCAAAGCCAGGGCACCCAGAGCCAA
GGTTCCGCCCTCCAGGACCCTGAGTGCTTCGCCCACCTGCTGCGA
TTCTACGACGGCATCTGCAAATGGGAGGAGGGCAGTCCCACGCCT
GTGCTGCATGTGGGCTGGGCCACCTTTCTTGTGCAGTCCCTAGGC
CGTTTTGAGGGACAGGTGCGGCAGAAGGTGCGCATAGTGAGCCGA
GAGGCCGAGGCGGCCGAGGCCGAGGAGCCGTGGGGCGAGGAAGCC
CGGGAAGGCCGGCGGCGGGGCCCACGGCGGGAGTCCAAGCCAGAG
GAGCCCCCGCCGCCCAAGAAGCCAGCACTGGACAAGGGCCTGGGC
ACCGGCCAGGGTGCAGTGTCAGGACCCCCCCGGAAGCCTCCTGGG
ACTGTCGCTGGCACAGCCCGAGGCCCTGAAGGTGGCAGCACGGCT
CAGGTGCCAGCACCCACAGCATCACCACCGCCGGAGGGTCCAGTG
CTCACTTTCCAGAGTGAGAAGATGAAGGGCATGAAGGAGCTGCTG
GTGGCCACCAAGATCAACTCGAGCGCCATCAAGCTGCAACTCACG
GCACAGTCGCAAGTGCAGATGAAGAAGCAGAAAGTGTCCACCCCT
AGTGACTACACTCTGTCTTTCCTCAAGCGGCAGCGCAAAGGCCTC
TGA
(SEQ ID NO: 1)

TABLE 2
Nucleic Acid Sequence of Codon-Optimized
Open Reading Frame Encoding Menin

ATGGGTTTGAAAGCGGCGCAGAAAACTCTCTTTCCCCTCAGGAGC
ATTGATGATGTTGTCAGATTGTTCGCAGCGGAACTCGGTCGCGAA
GAACCAGATCTCGTCCTACTAAGCCTCGTCCTTGGATTTGTTGAA
CACTTCTTGGCCGTGAATAGGGTGATTCCCACGAATGTGCCTGAA
TTGACGTTTCAACCGAGTCCTGCTCCAGATCCACCCGGTGGTTTG
ACTTATTTCCCTGTCGCTGATTTGTCCATTATTGCTGCACTGTAC
GCTAGGTTTACGGCTCAAATAAGAGGTGCAGTAGATCTCAGCCTG
TACCCCCGCGAGGGCGGCGTGAGTTCCCGCGAACTAGTTAAGAAG
GTATCTGACGTGATTTGGAATTCGCTGTCCCGTTCGTATTTTAAA
GACAGAGCTCATATTCAATCATTGTTTTCTTTTATTACTGGCTGG
TCACCGGTGGGTACTAAGCTGGATAGTTCGGGCGTCGCGTTCGCC
GTCGTGGGAGCTTGTCAAGCTCTCGGACTACGCGACGTGCATTTG
GCTCTCTCCGAAGACCACGCTTGGGTCGTCTTCGGTCCAAACGGA
GAACAAACCGCCGAAGTGACATGGCATGGAAAAGGAAATGAAGAT
CGACGAGGACAAACTGTGAACGCTGGCGTTGCAGAACGATCCTGG
CTTTATTTGAAGGGTTCGTATATGAGGTGCGATCGTAAAATGGAA
GTTGCCTTTATGGTATGCGCAATAAATCCGAGCATCGATTTGCAT
ACTGATTCTCTAGAACTATTGCAACTACAACAAAAGCTACTTTGG
CTATTGTACGATCTCGGTCACCTAGAGAGATATCCGATGGCACTG
GGAAATCTCGCTGACCTGGAAGAACTTGAACCTACTCCGGGACGT
CCGGATCCTTTGACATTGTATCATAAAGGAATCGCTTCCGCTAAA
ACTTATTACCGTGACGAGCATATTTATCCTTATATGTATCTCGCC
GGTTATCATTGCCGTAATAGGAACGTTCGTGAGGCGTTGCAAGCT
TGGGCTGATACCGCTACAGTGATTCAAGATTATAATTATTGTCGT
GAGGACGAAGAAATATATAAAGAATTCTTCGAGGTGGCTAACGAC
GTGATTCCTAATTTGCTTAAAGAAGCCGCATCCCTGCTTGAAGCT
GGAGAAGAACGTCCAGGAGAACAGTCGCAAGGTACGCAATCTCAG
GGAAGCGCTTTGCAAGATCCAGAATGTTTTGCTCATCTACTCAGA
TTTTATGATGGTATTTGTAAGTGGGAAGAAGGTTCCCCAACCCCC
GTCCTTCACGTCGGATGGGCTACATTCCTGGTTCAAAGCCTTGGA
AGGTTCGAAGGTCAAGTACGTCAGAAAGTTCGTATCGTCTCCAGG
GAAGCTGAAGCTGCTGAAGCTGAAGAACCTTGGGGTGAAGAGGCT
CGTGAGGGTCGTCGTCGCGGACCGCGCAGAGAATCGAAACCCGAA
GAACCGCCTCCTCCTAAGAAGCCTGCCCTTGATAAAGGATTGGGT
ACTGGTCAAGGCGCCGTCAGTGGTCCGCCTAGGAAACCACCAGGC
ACTGTAGCGGGAACTGCACGTGGTCCCGAAGGCGGTTCCACAGCC
CAAGTACCCGCTCCAACTGCTTCTCCACCGCCTGAAGGACCTGTA
CTAACCTTTCAATCTGAGAAAATGAAAGGAATGAAAGAACTCTTG
GTCGCAACAAAGATTAATTCCAGTGCAATAAAATTGCAGCTGACC
GCTCAATCACAGGTTCAAATGAAGAAGCAAAAGGTCAGCACACCA
TCAGATTATACCCTTAGTTTTCTGAAACGACAAAGGAAGGGACTT
TAA
(SEQ ID NO: 2)

TABLE 3
Amino Acid Sequence of Human Menin Protein

MGLKAAQKTLFPLRSIDDVVRLFAAELGREEPDLVLLSLVLGFVE
HFLAVNRVIPTNVPELTFQPSPAPDPPGGLTYFPVADLSIIAALY
ARFTAQIRGAVDLSLYPREGGVSSRELVKKVSDVIWNSLSRSYFK
DRAHIQSLFSFITGWSPVGTKLDSSGVAFAVVGACQALGLRDVHL
ALSEDHAWVVFGPNGEQTAEVTWHGKGNEDRRGQTVNAGVAERSW
LYLKGSYMRCDRKMEVAFMVCAINPSIDLHTDSLELLQLQQKLLW
LLYDLGHLERYPMALGNLADLEELEPTPGRPDPLTLYHKGIASAK
TYYRDEHIYPYMYLAGYHCRNRNVREALQAWADTATVIQDYNYCR
EDEEIYKEFFEVANDVIPNLLKEAASLLEAGEERPGEQSQGTQSQ
GSALQDPECFAHLLRFYDGICKWEEGSPTPVLHVGWATFLVQSLG
RFEGQVRQKVRIVSREAEAAEAEEPWGEEAREGRRRGPRRESKPE
EPPPPKKPALDKGLGTGQGAVSGPPRKPPGTVAGTARGPEGGSTA
QVPAPTASPPPEGPVLTFQSEKMKGMKELLVATKINSSAIKLQLT
AQSQVQMKKQKVSTPSDYTLSFLKRQRKGL*
(SEQ ID NO: 3)
Note that bolded and underlined amino acids
represent amino acids 160, 327, 331 and 349,
as disclosed herein.

“Gene” as used herein may be a natural (e.g., genomic) gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA, or antisense RNA. Those of ordinary skill in the art understand that the term “gene” can further incorporate gene regulatory sequences (e.g., promoters, enhancers, etc.) and/or intron sequences, and others of which are limited to coding sequences. It will further be appreciated that “gene” can include references to nucleic acids that do not encode proteins but rather encode functional RNA molecules such as tRNAs. For the purpose of clarity, as used herein, the term “gene” refers to a portion of a nucleic acid that encodes a protein; the term may optionally encompass regulatory sequences. For example, “gene” is not intended to exclude application of the term “gene” to non-protein coding expression units but rather to clarify that, in most cases, the term as used in this document refers to a protein coding nucleic acid.

Menin-inhibitory therapeutics or menin inhibitors can block interaction of menin protein with a rearranged MMLr/KMT2A protein. Menin-inhibitory therapeutics can displace Menin from chromatin. In menin-dependent cancers (e.g., acute leukemias of myeloid or lymphoblastic origin, like acute myeloid leukemia (AML or acute lymphoid leukemia (ALL)), patients having the cancer can be successfully treated with these menin inhibitors.

In some embodiments, the menin-inhibitory therapeutic comprises SNDX-5613, VTP-50469, MI-3454, MI-89, MI-3454, MCP-1, ML227, ML399, MIV-6, M-525, M-89, M-808, MI-2, MI-3, MI-2-2, MI-136, MI-463, MI-505, MI-538, BAY-155, MI-1481, KO-539 (Ziftomenib) or MI-3454.

In some embodiments, the menin-inhibitory therapeutic comprises JNJ-75276617, DSP-5336, DS-1594b or BMF-219.

In some cancer patients treated with menin-inhibitors, the cancers can become resistant or non-responsive to the drugs. As described herein, mutations in the gene encoding menin have been discovered that lead to or cause the resistance to menin inhibitors. Detection of these mutations can be diagnostic for resistance to the menin inhibitors. This application discloses the mutations and diagnostic methods for detecting the mutations in MEN1. In some embodiments, the subjects on whom the diagnostic tests are performed have been treated with a menin-inhibitory therapeutic. In some embodiments, the subject has relapsed, or the subject's cancer is not responsive to the menin-inhibitory therapeutic. Detection of MEN1 mutations in such patients can inform the physician that a change in treatment is warranted. In some embodiments, the diagnostic tests may be performed on a subject who has not yet been treated with a menin inhibitor. A diagnostic test on such a subject can provide information as to whether treatment of the subject with a menin inhibitor is appropriate.

“Mutation” refers to a change in the natural or reference nucleic acid sequence, including insertions, deletions, duplications, translocations, substitutions, frame shift mutations, silent mutations, nonsense mutations, missense mutations, point mutations, and base shift mutations, base conversion mutation, return mutation, micro satellite change, etc. In some embodiments, the amino acid sequence encoded by the nucleic acid sequence has at least one amino acid modification from the natural sequence.

The term “somatic mutation” or “somatic alteration” refers to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), copy number variations (CNVs), rearrangements, and splice variants. The presence of certain mutations can be associated with disease states (e.g., cancer, e.g., acute leukemias, e.g., Acute Myeloid Leukemia).

In certain embodiments, the somatic mutation is a silent mutation (e.g., a synonymous alteration). In other embodiments, the somatic mutation is a non-synonymous single nucleotide variant (SNV). In other embodiments, the somatic mutation is a passenger mutation (e.g., an alteration that has no detectable effect on the fitness of a clone). In certain embodiments, the somatic mutation is a variant of unknown significance (VUS), for example, a mutation, the pathogenicity of which can neither be confirmed nor ruled out. In certain embodiments, the somatic mutation has not been identified as being associated with a cancer phenotype.

In certain embodiments, the somatic mutation is not associated with, or is not known to be associated with, an effect on cell division, growth, or survival. In other embodiments, the somatic mutation is associated with an effect on cell division, growth, or survival.

In some embodiments, the MEN1 mutations are somatic mutations. In some embodiments, MEN1 mutations can be in the germ line or germline. In certain embodiments, the germline mutation is a SNP, a base substitution, an insertion, a deletion, an indel, or a silent mutation (e.g., synonymous mutation).

In some embodiments, the mutations in the MEN1 gene can be any mutation that results in resistance of a cell to a menin inhibitor. In some embodiments, such mutations in the MEN1 gene can substitute an amino acid for a wild-type amino acid in menin protein at positions 327, 331, 349, 160, or combinations thereof. In some embodiments, the wild-type amino acid at position 327 comprises methionine. In some embodiments, the wild-type amino acid at position 331 comprises glycine. In some embodiments, the wild-type amino acid at position 349 comprises threonine. In some embodiments, the wild-type amino acid at position 160 comprises serine. In some embodiments, the wild-type amino acid is substituted with an isoleucine, valine, arginine, methionine, aspartic acid, or cysteine. In some embodiments, the wild-type amino acid is substituted with an isoleucine or valine at position 327. In some embodiments, the wild-type amino acid is substituted with an arginine at position 331. In some embodiments, the wild-type amino acid is substituted with an aspartic acid at position 331. In some embodiments, the wild-type amino acid is substituted with a methionine at position 349. In some embodiments, the wild-type amino acid is a substituted with a cysteine at position 160.

In some embodiments, the mutation in the MEN1 gene substitutes the wild-type methionine with isoleucine or valine at position 327. In some embodiments, the mutation in the MEN1 gene substitutes the wild-type glycine with arginine or aspartic acid at position 331. In some embodiments, the mutation in the MEN1 gene substitutes the wild-type threonine with methionine at position 349. In some embodiments, the mutation in the MEN1 gene substitutes the wild-type serine with cysteine at position 160.

In some embodiments, the mutation in the MEN1 gene substitutes the wild-type methionine with isoleucine or valine at position 327 and substitutes the wild-type glycine with arginine at position 331. In some embodiments, the mutation in the MEN1 gene substitutes the wild-type methionine with isoleucine at position 327 and substitutes the wild-type glycine with arginine at position 331.

In some embodiments, substitution of the wild-type amino acid at position 349 occurs in cancers that have substitutions of the wild-type amino acid at positions 327, 331, or both 327 and 331.

In some embodiments, mutations that contribute to resistance to menin inhibitors may be in genes other than MEN1. In some embodiments, those mutations may work in concert with MEN1 mutations to contribute to a cancer phenotype. In some embodiments, mutations in MEN1 alone can result in resistance to menin inhibitors.

A functional mutation is a mutation that, compared with a reference sequence (e.g., a wild-type or unmutated sequence) has an effect on cell division, growth, or survival (e.g., promotes cell division, growth, or survival). In certain embodiments, the functional alteration is identified as such by inclusion in a database of functional mutations, e.g., the COSMIC database (see Forbes et al., Nucl. Acids Res. 43 (D1): D805-D811, 2015, which is herein incorporated by reference in its entirety). In other embodiments, the functional mutation is a mutation with known functional status (e.g., occurring as a known somatic alteration in the COSMIC database). In certain embodiments, the functional mutation is a mutation with a functional status (e.g., a truncation in a tumor suppressor gene). In certain embodiments, the functional mutation is a driver mutation (e.g., a mutation that gives a selective advantage to a clone in its microenvironment. e.g., by increasing cell survival or reproduction, like a menin-inhibitor-resistant cell in a cancer tissue in a subject being treated with a menin inhibitor). In other embodiments, the functional mutation is a mutation that can cause clonal expansions. In certain embodiments, the functional mutation is a mutation that can cause one, two, three, four, five, or all six of the following: (a) self-sufficiency in a growth signal; (b) decreased, e.g., insensitivity, to an antigrowth signal; (c) decreased apoptosis; (d) increased replicative potential; (e) sustained angiogenesis; or (f) tissue invasion or metastasis.

“Polynucleotide,” or nucleic acid,” as used interchangeably herein, refer to polymers of nucleotides of any length, and include DNA and RNA. The nucleotides can be deoxyribonucleotides, ribonucleotides, modified nucleotides, or bases, and/or their analogs, or any substrate that can be incorporated into a polymer by DNA or RNA polymerase, or by a synthetic reaction. Thus, for instance, polynucleotides can include, without limitation, single- and double-stranded DNA, DNA including single- and double-stranded regions, single- and double-stranded RNA, and RNA including single- and double-stranded regions, hybrid molecules comprising DNA and RNA that may be single stranded or, for example, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but can involve only a region of some of the molecules. One of the molecules of a triple-helical region often is an oligonucleotide. The term “polynucleotide” specifically includes cDNAs.

“Oligonucleotide,” as used herein, refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

A “disorder” is any condition that can benefit from treatment including, but not limited to, chronic and acute disorders or diseases including those pathological conditions which predispose the mammal to the disorder in question.

The terms “cancer” and “cancerous” refer to or describe the physiological condition in mammals that is characterized by unregulated cell growth. By “early-stage cancer” or “early-stage tumor” can refer to a cancer that is not invasive or metastatic or is classified as a Stage 0, 1, or 2 cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. A nonlimiting list of cancers for which the methods disclosed herein include leukemia (e.g., Acute Myeloid Leukemia), bladder, breast, colon and rectal, endometrial, kidney, liver, lung, lymphoma (e.g., non-Hodgkin lymphoma), melanoma, pancreatic, prostate, thyroid, and others. The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

In the case of blood cancers, blood primarily consists of red blood cells (RBC), white blood cells (WBC) and platelets. Red blood cells carry oxygen to the body, the white blood cells police and protect the body, and platelets help clot the blood when there is injury. Abnormalities in these cell types can lead to blood cancer. The main categories of blood cancer are Acute Lymphocytic or Lymphoblastic Leukemias (ALL), Chronic Lymphocytic or Lymphoblastic Leukemias (CLL), Acute Myelogenous or Myeloid Leukemias (AML), and Chronic Myelogenous or Myeloid Leukemias (CML).

Both leukemia and lymphoma are hematologic malignancies (cancers) of the blood and bone marrow. In the case of leukemia, the cancer is characterized by abnormal proliferation of leukocytes and is one of the four major types of cancer. The cancer interferes with the body's ability to make blood, and the cancer attacks the bone marrow and the blood itself, causing fatigue, anemia, weakness, and bone pain. Leukemia is diagnosed with a blood test in which specific types of blood cells are counted; it accounts for about 29,000 adults and 2,000 children diagnosed each year in the United States. Treatment for leukemia can include chemotherapy and radiation to kill the cancer and may involve bone marrow transplantation in some cases.

Leukemias are classified according to the type of leukocyte most prominently involved. Acute leukemias are predominantly undifferentiated cell populations and chronic leukemias have more mature cell forms. The acute leukemias are divided into lymphoblastic (ALL) and non-lymphoblastic (ANLL) types, with ALL being predominantly a childhood disease while ANLL, also known as acute myeloid leukemia (AML), being a more common acute leukemia among adults.

AML is characterized by an increase in the number of myeloid cells in the marrow and an arrest in their maturation, frequently resulting in hematopoietic insufficiency. In the United States, the annual incidence of AML is approximately 2.4 per 100,000 and it increases progressively with age to a peak of 12.6 per 100,000 adults 65 years of age or older. Despite improved therapeutic approaches, prognosis of AML is very poor around the globe. Even in the United States, five-year survival rate among patients who are less than 65 years of age is less than 40%.

Acute myeloid leukemia (AML) is a heterogeneous disorder that includes many entities with diverse genetic abnormalities and clinical features. The pathogenesis is known for relatively few types of leukemia. Patients with intermediate and poor risk cytogenetics represent the majority of AML; chemotherapy-based regimens fail to cure most of these patients and stem cell transplantation is frequently the treatment choice. Since allogeneic stem cell transplantation is not an option for many patients with high-risk leukemia, there is a need to improve our understanding of the biology of these leukemias and to develop improved therapies. Despite considerable advances, not enough is known of the etiology, cell physiology and molecular genetics of acute myeloid leukemia. As such, the development of effective new agents and new treatment and/or prognostic methods against myeloid leukemia, and for example, acute myeloid leukemia, remains a focal point today in translational oncology research.

Significant progress has been made in understanding risk factors, including genetic factors, that may contribute to AML, but the relevance of these factors to clinical outcome remains unclear. In addition, the expression level and antibody staining pattern of several proteins have been shown to be predictive of outcome and of response to therapy. However, the clinical outcome of individual patients remains uncertain, and the ability to predict which patients can benefit from a certain type of therapy (e.g., a certain drug or class of drug) remains elusive.

The terms “subject” and “patient,” as used interchangeably herein, refer to any animal including, but not limited to, humans, non-human primates, bovines, equines, felines, canines, pigs, rodents (e.g., mice), and the like. A subject to be treated or tested for responsiveness to a treatment (e.g., revumenib) according to the methods described herein may be one who has been diagnosed with a cancer, such as those described herein, e.g., acute myeloid leukemia. Diagnosis may be performed by any method or techniques known in the art, such as x-ray, MRI, or biopsy, and confirmed by a physician. To minimize exposure of a subject to drug treatments that may not be therapeutic, the patient may be determined to be either responsive or nonresponsive to a cancer treatment, such as revumenib (i.e., SNDX-5613), according to the methods described herein.

As used herein, the terms “subject at risk for cancer” or “subject at risk for leukemia” refer to a subject with one or more risk factors for developing cancer and/or leukemia. Risk factors include, but are not limited to, gender, age, genetic predisposition, environmental exposure, and previous incidents of cancer, preexisting non-cancer diseases, and lifestyle.

The term “sample.” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that can be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof. The sample may be in vitro or in vivo.

“Tissue sample” or “cell sample” refers to a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells, and non-cancerous cells). The tissue sample may contain compounds which are not naturally intermixed with the tissue in nature such as preservatives, anticoagulants, buffers, fixatives, nutrients, antibiotics, or the like. In some instances, the tissue sample or tumor tissue sample is not a blood sample or sample or a blood constituent, such as plasma.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein, a “section” of a tissue sample can refer to a single part or piece of a tissue sample, for example, a thin slice of tissue or cells cut from a tissue sample (e.g., a tumor sample). For example, multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

“Resistant” or “resistance” as used herein refers to a cell (e.g., a cancer cell), a tissue (e.g., a tumor), or a patient having cancer (e.g., a human having cancer) that can withstand treatment with an anti-cancer agent (e.g., revumenib). For example, in resistance cells, the treatment does not inhibit the growth of a cancer cell in vitro by about 70%, 80%, 90%, 95%. 99% or 100% relative to the growth of a cancer cell not exposed to the treatment.

A cancer patient (e.g., a patient with acute myeloid leukemia) may also have resistance to a cancer therapy other than revumenib, such as surgery, radiation, or a therapeutic agent (e.g., docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, or 5-fluorouracil (5-F)).

### Methods

In various embodiments, resistance of a cancer to a menin-inhibitor is diagnosed or determined. In some embodiments, samples comprising cancer cells are obtained from a patient. In some embodiments, the cancer cells are tested for the presence of a mutation in a MEN1 gene. In some embodiments, the sample comprises blood, bone marrow, or spinal fluid. In some embodiments, the patient has acute myeloid leukemia.

The term “diagnose” as used herein refers to the act or process of identifying or determining a disease or condition in a mammal or the cause of a disease or condition by the evaluation of the signs and symptoms of the disease or disorder. For example, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to said particular disease: i.e., there may be differential diagnoses that can be inferred from a diagnostic factor or symptom. Likewise, there may be instances where a factor or symptom that is indicative of a particular disease is present in an individual that does not have the particular disease.

The term “diagnosis” is used herein to refer to the identification or classification of a molecular or pathological state, disease, or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a certain type of symptom or condition of a disease or disorder (e.g., cancer). For example, a method of aiding diagnosis of a disease or condition (e.g., cancer) can comprise measuring certain somatic mutations in a biological sample from an individual.

“Prognosis” as used herein refers to a forecast as to the probable outcome of cancer, including the prospect of recovery from the cancer. As used herein the terms prognostic information and predictive information are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a therapy (wherein response can be described in any of a variety of ways). Prognostic and predictive information are included within the broad category of diagnostic information.

A prognosis of a patient can be made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis can be expressed as the amount of time a patient can be expected to survive. Alternatively, a prognosis can refer to the likelihood that the disease goes into remission or to the amount of time the disease can be expected to remain in remission. Prognosis can be expressed in various ways; for example, prognosis can be expressed as a percent chance that a patient will survive after one year, five years, ten years, or the like. Alternatively, prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The terms “favorable prognosis” and “positive prognosis,” or “unfavorable prognosis” and “negative prognosis” as used herein are relative terms for the prediction of the course and/or outcome of a condition or a disease. A favorable or positive prognosis predicts a better outcome for a condition than an unfavorable or negative or adverse prognosis. For example, a “favorable prognosis” is an outcome that is relatively better than many other prognoses that could be associated with a particular condition, whereas an “unfavorable prognosis” predicts an outcome that is relatively worse than many other prognoses that could be associated with a particular condition. Examples of a favorable or positive prognosis include a better than average cure rate, a lower propensity for metastasis, a longer than expected life expectancy, differentiation of a benign process from a cancerous process, and the like. For example, if a prognosis is that a patient has a 50% probability of being cured of a particular cancer after treatment, while the average patient with the same cancer has only a 25% probability of being cured, then that patient exhibits a positive prognosis. A positive prognosis may be diagnosis of a benign tumor if it is distinguished over a cancerous tumor.

The term “relapse” or “recurrence” as used in the context of cancer herein refers to the return of signs and symptoms of cancer after a period of remission or improvement. In some embodiments, a patient having a cancer that was successfully treated with a menin inhibitor, but the cancer later became resistant to inhibition by the menin inhibitor, can be said to be relapsed.

As used herein a “response” to treatment may refer to any beneficial alteration in a subject's condition that occurs as a result of treatment. Such alteration may include stabilization of the condition (e.g., prevention of deterioration that can take place in the absence of the treatment), amelioration of symptoms of the condition, improvement in the prospects for cure of the condition. One may refer to a subject's response or to a tumor's response These concepts can be used interchangeably herein.

“Expression profile” as used herein can refer to a genomic expression profile. Profiles can be generated by any convenient means for determining a level of a nucleic acid sequence e.g., quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art.

“Microarray” refers to an ordered arrangement of hybridizable array elements, such as polynucleotide probes, on a substrate.

The terms “level of expression” or “expression level” can be used interchangeably and can refer to the amount of a biomarker in a biological sample. “Expression” refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker).

“Amplification,” as used herein refers to the process of producing multiple copies of a desired sequence. “Multiple copies” can refer to at least two copies. A “copy” does not necessarily refer to perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

In some embodiments, nucleic acids in cancer cells must undergo amplification in order to be tested for the presence of a mutation in a MEN1 gene. In some embodiments, the nucleic acid amplification may include polymerase chain reaction (PCR), reverse-transcription PCR, quantitative PCR, real-time PCR, isothermal amplification, linear amplification, or isothermal linear amplification, quantitative fluorescent PCR (QF-PCR), multiplex fluorescent PCR (MF-PCR), single cell PCR, restriction fragment length polymorphism PCR (PCR-RFLP), PCR-RFLP/RT-PCR-RFLP, hot start PCR, nested PCR, in situ colony PCR, in situ rolling circle amplification (RCA), bridge PCR (bPCR), picotiter PCR, digital PCR, droplet digital PCR, or emulsion PCR (emPCR). Other suitable amplification methods include ligase chain reaction (LCR (oligonucleotide ligase amplification (OLA)), transcription amplification, cycling probe technology (CPT), molecular inversion probe (MIP)PCR, self-sustained sequence replication, selective amplification of target polynucleotide sequences, consensus sequence primed polymerase chain reaction (CP-PCR), arbitrarily primed polymerase chain reaction (AP-PCR), transcription mediated amplification (TMA), degenerate oligonucleotide-primed PCR (DOP-PCR), multiple-displacement amplification (MDA), strand displacement amplification (SDA), and nucleic acid based sequence amplification (NABS A).

The technique of “polymerase chain reaction” or “PCR” as used herein refers to a procedure wherein minute amounts of a specific piece of nucleic acid, RNA and/or DNA, are amplified as described, for example, in U.S. Pat. No. 4,683,195. Sequence information from the ends of the region of interest or beyond needs to be available, such that oligonucleotide primers can be designed: these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51: 263 (1987) and Erlich, ed. PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a certain nucleic acid.

The term “multiplex-PCR” refers to a single PCR reaction carried out on nucleic acid obtained from a single source (e.g., an individual) using more than one primer set for the purpose of amplifying two or more DNA sequences in a single reaction.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164 (1): 35-42 (2004) and Ma et al., Cancer Cell 5: 607-616 (2004).

In some embodiments, nucleic acid amplification includes digital PCR. It will be appreciated that digital PCR may include any method, process, and/or protocol, using instruments and/or kits associated with performing such, that can discretely amplify and quantitate a nucleic acid(s) within individual partitions of a sample. In some embodiments, the individual partitions for a digital PCR may be generated by a microfluidic process, such as by using a microfluidic device, and/or by a droplet generating process. Generation of individual partitions by a microfluidic process, such as by using a microfluidic device, and/or a droplet generating process to provide a plurality of partitions in the form of droplets and performing nucleic acid amplification thereon has been described in the art as “droplet digital PCR.” The droplets generated for droplet digital PCR may be provided in, for example, a water-in-oil emulsion. In some embodiments, the methods, processes, and/or protocols, and instruments and/or kits for performing nucleic acid amplification on partitions in the form of droplets generated using a microfluidic device/process and/or a droplet generating process, are commercially available, for example, but not limited to, those provided by Bio-Rad, 10× Genomics, Qiagen, and/or ThermoFisher. In an exemplary embodiment, nucleic acid amplification includes droplet digital PCR (ddPCR™) using Bio-Rad's QX100™ or QX200™ Droplet Digital PCR systems, and analysis of nucleic acid amplification products produced by the same but is not limited thereto.

In some embodiments, the cancer cells can undergo sequencing of nucleic acids contained in the cells in order to be tested for the presence of a mutation in a MEN1 gene. In some embodiments, the cancer cells undergo targeted sequencing.

“Sequencing”. “sequence determination” and the like refers to any and all biochemical methods that may be used to determine the order of nucleotide bases in a nucleic acid. Targeted sequencing can include the ability to detect complex variation, avoiding clonal errors, and analysis that is less computationally burdensome (e.g. de novo sequencing). There are several embodiments of targeted sequencing.

The term “targeted sequencing” refers to efficient sequencing of a small subset of the genome. In clinical settings, sequencing a subset of the genome not only reduce costs, but also focuses on the relevant regions. The main challenge for clinical targeted resequencing methods is obtaining complete and uniform coverage of all target regions. Some popular methods for target enrichment rely on lengthy and inefficient hybrid capture or multiplexed PCR techniques, resulting in lower coverage and more off-target sequencing reads.

The terms “whole genome sequencing,” “full genome sequencing”, “complete genome sequencing”, and “entire genome sequencing” refer to a laboratory process that determines the complete DNA sequence of an organism's genome at a single time. This entails sequencing all of an organism's chromosomal DNA as well as DNA contained in the mitochondria and, for plants, in the chloroplast.

For example, “correlate” or “correlating” can refer to comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues: (4) inhibition (i.e. reduction, slowing down, or complete stopping) of metastasis: (5) relief, to some extent, of one or more symptoms associated with the disease or disorder (e.g., cancer); (6) increase or extension in the length of survival, including overall survival and progression free survival; and/or (7) decreased mortality at a given point of time following treatment. An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival): resulting in an objective response (including a complete response or a partial response); or improving signs or symptoms of cancer.

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of cancer (e.g., disappearance of all target lesions) in response to treatment. This does not always mean the cancer has been cured.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration at least the same as the treatment duration, at least 1.5×, 2.0×, 2.5×, or 30× length of the treatment duration, or longer.

As used herein, “reducing or inhibiting cancer relapse” can refer to reducing or inhibiting tumor or cancer relapse or tumor or cancer progression. As described herein, cancer relapse and/or cancer progression include(s), without limitation, cancer metastasis.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

The term “survival” refers to the patient remaining alive and includes overall survival as well as progression free survival.

As used herein, “progression-free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” or “OS” refers to the percentage of individuals in a group who are likely to be alive after a certain duration of time.

In various embodiments, a cancer patient who has been previously treated with a menin-inhibitory therapeutic is tested for presence of a mutation in the MEN1 gene. In some embodiments, samples comprising cancer cells are obtained from a patient. In some embodiments, the cancer cells are tested for the presence of a mutation in a MEN1 gene. In some embodiments, the sample comprises blood, bone marrow, or spinal fluid. In some embodiments, the patient has acute myeloid leukemia. In some embodiments, treatment with the menin-inhibitory therapeutic is discontinued if a mutation in a MEN1 gene is detected. In some embodiments, treatment with a different menin inhibitor, or treatment with a therapeutic that is not a menin inhibitor can be initiated. In some embodiments, the patient may be administered a therapy that is not a drug. In some embodiments, different therapies may be simultaneously administered to the patient.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The term “therapeutically effective amount” refers to an amount of a drug effective to treat a disease or disorder in a mammal. In the case of cancer, the therapeutically effective amount of the drug may reduce the number of cancer cells; reduce the tumor size: inhibit (i.e., slow to some extent and in other instances, stop) cancer cell infiltration into peripheral organs; inhibit (i.e., slow to some extent and in other instances, stop) tumor metastasis; inhibit, to some extent, tumor growth; and/or relieve to some extent one or more of the symptoms associated with the disorder.

The invention provides for both prophylactic and therapeutic methods of treating a subject at risk of (or susceptible to) a cancer (for example, if an early detection cancer biomarker is identified in such a subject), or other cell proliferation-related diseases or disorders. Such diseases or disorders include but are not limited to, e.g., those diseases or disorders associated with aberrant expression of a MEN1 mutation. For example, the methods are used to treat, prevent, or alleviate a symptom of cancer. In an embodiment, the methods are used to treat, prevent, or alleviate a symptom of acute myeloid leukemia. Nonlimiting examples of other cancers that can be treated by compositions described herein comprise lung cancer, ovarian cancer, prostate cancer, colon cancer, cervical cancer, brain cancer, skin cancer, liver cancer, pancreatic cancer, or stomach cancer. Additionally, the methods of the invention can be used to treat hematologic cancers such as leukemia and lymphoma. Alternatively, the methods can be used to treat, prevent, or alleviate a symptom of a cancer that has metastasized. For example, cancers that can be treated or prevented or for which symptoms can be alleviated include B-cell chronic lymphocytic leukemia (CLL), non-small-cell lung cancer, melanoma, ovarian cancer, lymphoma, or renal-cell cancer. Cancers that can also be treated or prevented or for which symptoms can be alleviated include those solid tumors with a high mutation burden and WBC in filtrate.

Accordingly, in one aspect, the invention provides methods for preventing, treating, or alleviating a symptom of cancer or a cell proliferative disease or disorder in a subject by discontinuing treatment with the menin-inhibitory therapeutic if a mutation in a MEN1 gene is detected. In some embodiments, if one or more mutations disclosed herein are detected in cancer cell genomes of a patient, the menin-inhibitory therapy can be discontinued. In some embodiments, the therapy may be discontinued because it is no longer effective or efficacious.

The term “concurrently” is used herein to refer to administration of two or more therapeutic agents, where at least part of the administration overlaps in time. Accordingly, concurrent administration includes a dosing regimen when the administration of one or more agent (s) continues after discontinuing the administration of one or more other agents).

For example, “reduce or inhibit” can refer to causing an overall decrease of 20%, 30%, 40%, 50%, 50%, 70%, 75%, 80%, 85%, 90%, 95%, or greater. Reduce or inhibit can refer, for example, to the symptoms of the disorder being treated, the presence or size of metastases, or the size of the primary tumor.

In some embodiments, reagents useful for detecting mutations in MEN1 can be packaged and sold in a kit. In some embodiments, in addition to the reagents, the kit may include a package insert. The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., MEN1) described herein. In certain embodiments, the manufacture or kit is promoted, distributed, or sold as a unit for performing the methods described herein.

The phrase “based on” when used herein can refer to information about one or more biomarkers used to inform a treatment decision, information provided on a package insert, or marketing promotional guidance, etc.

The presence and/or levels (amount) of somatic mutations can be determined qualitatively and/or quantitatively based on any suitable criterion known in the art, including but not limited to the measurement of DNA. mRNA, DNA, proteins, protein fragments, and/or gene copy number levels in an individual. In some instances, a comprehensive genomic profile of an individual is determined. In some instances, a comprehensive genomic profile of a sample (e.g., tissue sample, formalin-fixed, paraffin embedded (FFPE) tissues sample, core, or fine needle biopsies) collected from an individual is determined. In some instances, the determination of the genomic profile comprises applying next-generation sequencing methods, known in the art, or described herein, to identify genomic alterations (e.g., somatic mutations (e.g., base substitutions, insertions, and deletions (indels), copy number alterations (CNAs) and rearrangements)) known to be unambiguous drivers of cancer (e.g., solid tumors).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Acquired resistance to Menin-MLL-inhibitors is a phenomenon that was recently identified as a therapeutic challenge in a phase-1 clinical trial probing the compound SNDX5613 in patients with AML. We identified and characterized point mutations within the Menin (MEN1) coding sequence (M327I, M327V, G331R, T349M), that occur during or after treatment and significantly decrease drug sensitivity. Knowledge about these mutations can be used to identify a genetic cause for treatment failure during Menin-MLL-inhibitor treatment in the clinical setting. This identification can facilitate better treatment options for patients.

This is the first distinct mechanism of resistance towards Menin-MLL1-inhibitors discovered. No other studies have yet provided a genetic rationale to stratify patients that developed drug resistance to SNDX5613 or similar molecules.

This approach can be used to develop targeted sequencing and or quantitative real-time PCR approaches to monitor the occurrence of a set of somatic mutations in patients under Menin-inhibitor treatment. Since the discovered mutations are strongly associated with drug resistance, this approach can help with early identification of treatment failure in patients before they clinically relapse.

This approach can be used in concert with other diagnostic screening tools (e.g., Heme-panel sequencing). Furthermore, the approach can be used to evaluate the success of combination therapy approaches to prevent outgrowth of mutated clones and/or target the mutated clones that lose sensitivity to Menin-inhibitor monotherapy.

### Example 2

### Acquired MEN1 Mutations Mediate Resistance to Menin Inhibition

Chromatin binding proteins are regulators of cell state in hematopoiesis^{1,2}. Acute leukemias driven by rearrangements of the Mixed Lineage Leukemia gene (KMT2Ar) or Nucleophosmin (NPM1c) mutations require the chromatin adapter protein Menin, encoded by the MEN1 gene, to sustain aberrant leukemogenic gene expression programs³⁻⁵. In a phase 1 first-in-human clinical trial, the Menin-inhibitor SNDX-5613 (revumenib), designed to disrupt the Menin-MLL1 interaction, induced clinical responses in leukemia patients with KMT2Ar or mutant NPM1⁶. We identified somatic mutations in MEN1 at the SNDX-5613/Menin interface in patients with acquired resistance to Menin inhibition. Consistent with the genetic data in patients, inhibitor/Menin interface mutations represent a conserved mechanism of therapeutic resistance in xenograft models and in an unbiased base-editor screen. These mutants attenuate drug-target binding by generating structural perturbations that impact small molecule binding but not the interaction with the natural ligand MLL1 and prevent inhibitor-induced eviction of Menin and MLL1 from chromatin. This study is the first to demonstrate that a chromatin-targeting therapeutic exerts sufficient selection pressure to drive evolution of escape mutants leading to sustained chromatin occupancy as a common mechanism of therapeutic resistance.

Menin is a chromatin adaptor protein that is involved in the formation and stability of highly conserved multiprotein complexes on chromatin, including Mixed Lineage Leukemia 1 (MLL1; KMT2A) and MLL2 (KMT2B) histone methyltransferase complexes and the JUND transcription factor complex^{4,7,8}. Menin is involved in the development and maintenance of acute leukemias driven by rearrangements involving MLL1 (KMT2Ar) or truncating mutations of the Nucleophosmin gene (NPM1c)^{3,5}. A series of small molecule inhibitors that disrupt the Menin-MLL1 protein-protein interaction have been developed^{3,9-12} and demonstrate potent activity in pre-clinical models, including the ability to eradicate disease^{3,9,13}. Based on this, several Menin-inhibitors recently entered phase 1 clinical trials (NCT04065399, NCT04067336, NCT04811560, NCT05388903, NCT04988555, NCT05153330). The Menin inhibitor SNDX-5613 has been reported to be safe and effective in patients with relapsed or refractory acute leukemia with an KMT2Ar or NPM1c mutation. In the ongoing phase 1/2 first-in-human study (AUGMENT-101) of SNDX-5613, patients with KMT2Ar or NPM1c-mutant leukemia had an overall response rate of 53% with 30% of treated patients achieving a complete remission (CR) or complete remission with partial hematologic recovery (CRh)⁶. Here, we identified and characterized somatic mutations within the MEN1 gene that arise during Menin-inhibitor treatment in model systems and on the AUGMENT-101 trial (NCT04065399) which mediate therapeutic resistance.

Somatic MEN1 Mutations in Patients with Menin-Inhibitor Acquired Resistance

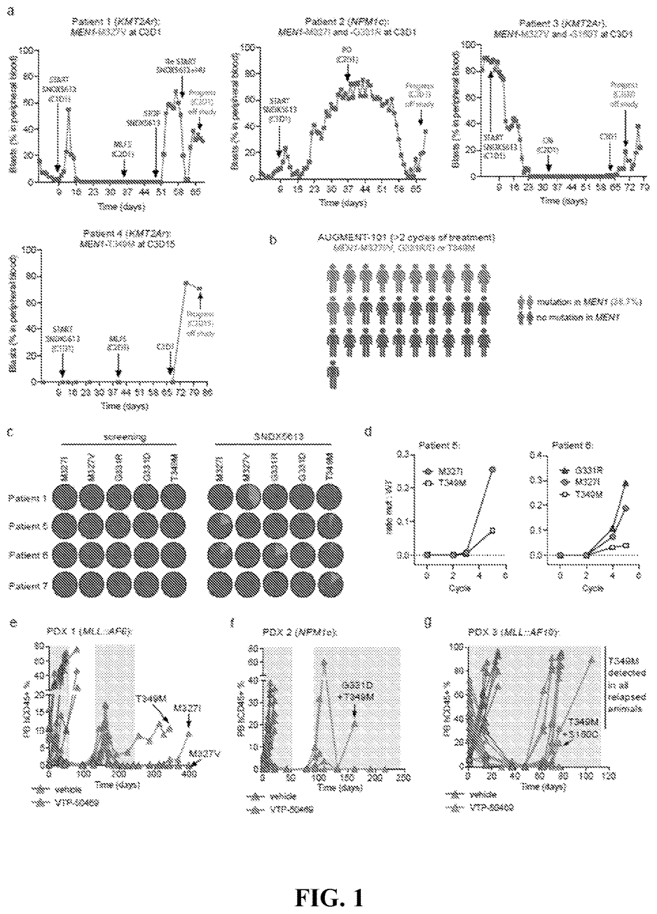

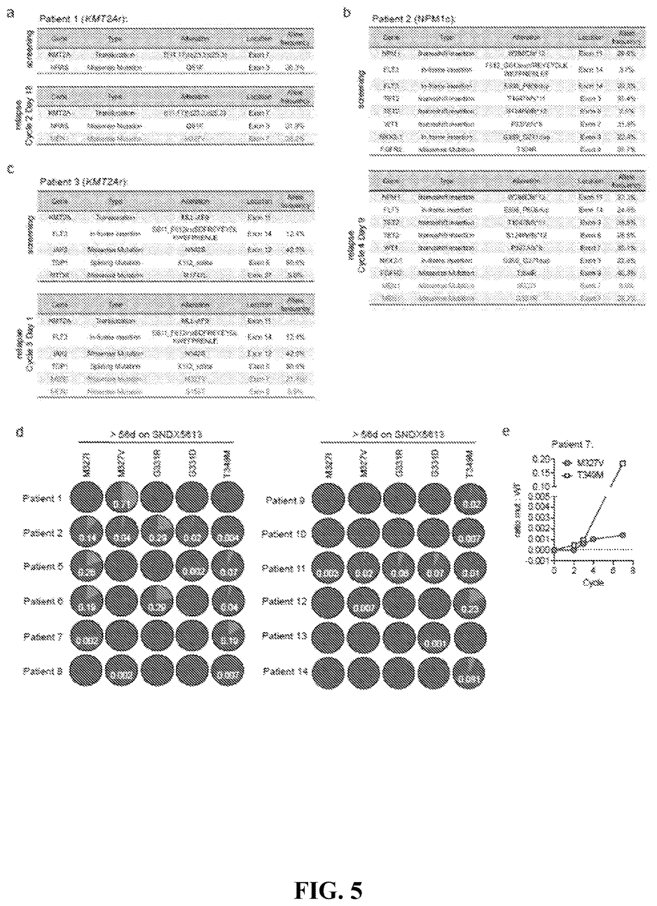

Despite significant single-agent activity of SNDX-5613 in a heavily pretreated KMT2Ar and NPM1c-mutant leukemia patient population, we identified patients who were treated on the AUGMENT-101 phase 1 study and subsequently developed acquired resistance following an initial response (FIG. 1A). Patients 1 and 4 with relapsed KMT2Ar AML achieved morphologic leukemia-free states (MLFS) after one cycle of treatment, followed by relapse despite continued exposure to study drug. Patient 2 with NPM1c-mutant AML had a reduction in circulating blast counts after 2 cycles of SNDX-5613 followed by disease progression, while Patient 3 with KMT2Ar AML achieved a complete remission with incomplete count recovery (CR) and then progressed despite continued Menin-inhibitor treatment (FIG. 1A). Next-generation targeted sequencing of bone marrow specimens from these patients at diagnosis revealed a largely stable landscape of well characterized leukemia drivers but somatic mutations within the MEN1 gene were detected at time of relapse on SNDX-5613 (FIG. 5A-C). In 3/4 patients the M327-residue was affected (M327V or M327I) and T349M, G331R and S160T were detected in each one of the patients at the end of treatment, respectively (FIG. 1A). Mutation allele frequencies (MAF) ranged from 5.9% to 28.2% (FIG. 5A-C), owing to dilution effects from normal leukocytes.

To quantitatively assess the frequency of these new somatic mutations within the population of KMT2Ar and NPM1c-mutant patients that had been exposed to SNDX-5613, we performed droplet digital PCR (ddPCR) for MEN1-M327V, -M327I, -G331R, -G331D and -T349M on DNA samples collected during the AUGMENT-101 phase-1 trial at different study centers. We identified 31 patients who were treated with SNDX-5613 for more than 2 cycles of treatment (>56 days) and had DNA material available for analysis. Among these patients, 12 individuals (38.7%) carried one or more MEN1− mutations (FIG. 1B, FIG. 5D). While not wishing to be bound by theory, these mutations were not detected in the pre-treatment samples by ddPCR, indicating they were either not present or in very low abundance until Menin-inhibition established a selective fitness advantage (FIG. 1C). Again, not wishing to be bound by theory, longitudinal assessment of the detected mutations in single patients confirmed clonal outgrowth during drug treatment and indicated that selection for MEN1-mutant leukemia cells occurs after approximately 2 cycles of therapy (FIG. 1D, FIG. 5E).

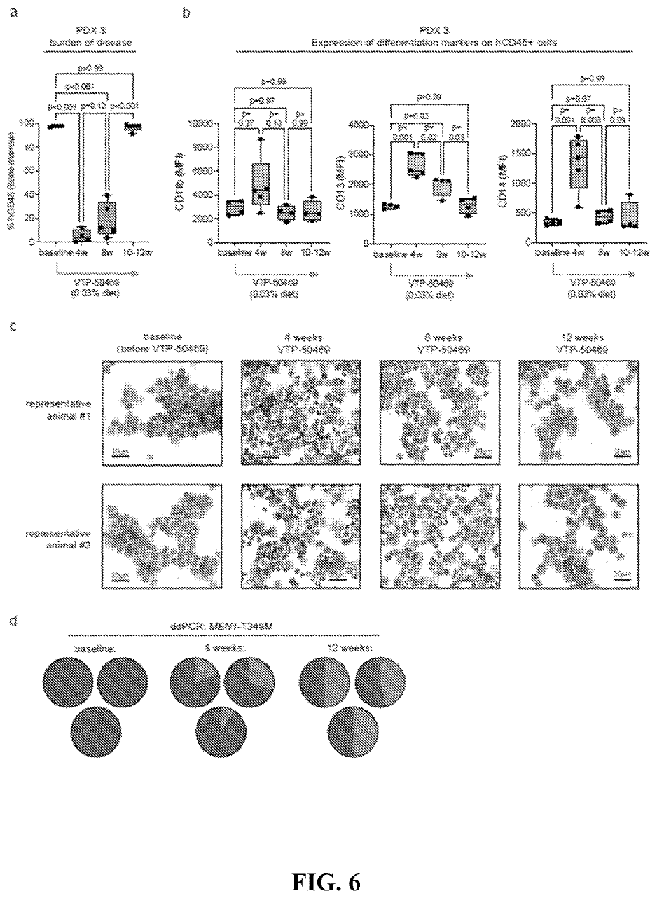

In parallel, we conducted a pre-clinical study in which we treated 5 different patient-derived xenografts (PDX) derived from AML patients (4×KMT2Ar, 1×NPM1c) with the Menin-inhibitor VTP-50469, a close analog of SNDX-5613 that binds similarly to Menin. We assessed long-term survival for up to 400 days after initiation of treatment. Out of 45 animals treated with the drug, 22 xenografted mice (from all 5 PDX models) relapsed during drug exposure or after treatment cessation or had human leukemia cells detected in the bone marrow at the end of treatment without overt manifestations of disease. Targeted DNA-sequencing and/or ddPCR was performed on 33 specimens (11 untreated and all 22 relapsed animals). MEN1 mutations were identified in 15 (68%) of those relapsed samples from 4 PDX models (FIG. 1E-G). MEN1-M327V, -M327I, -G331R, -G331D, -T349M and -S160C mutations were detected, demonstrating high recurrence and a predictive value of the PDX model system. In PDX1 (MLL::AF6) and PDX2 (NPM1c) we observed relapse in single animals, while disease was eradicated in a large proportion of the cohort (FIG. 1E, F). The MEN1 mutations identified in those animals were diverse and included M327I, M327V, G331D as well as T349M and arose after a long period of drug exposure, and while not wishing to be bound by theory, indicating de novo mutations were acquired during treatment. In contrast, all Menin-inhibitor treated recipients engrafted with sample PDX3 (MLL::AF10) relapsed after about 2 months of continuous drug treatment (FIG. 1G, FIG. 6A-C). All mice possessed T349M mutations and, while not wishing to be bound by theory, reflecting the selection of a pre-existent ultra-low frequency variant that could not be detected pre-therapy in bone marrow isolates (FIG. 1G, FIG. 6D).

Interestingly, in PDX4 (MLL::AF9, AML), resistance developed in 4/9 animals without MEN1 mutations or other newly acquired genetic drivers detectable, indicating that adaptation and non-genetic processes may also lead to Menin-inhibitor resistance in individual cases. Similarly, disease persistence could be detected in 4/9 VTP-50469 treated animals in PDX5 (MLL::AF9, AML), while in only one of these animals a Menin mutation (T349M) could be detected.

In summary, we identified recurrent somatic mutations within the MEN1-gene which affect the M327-, G331-, T349- and S160-residues of Menin in patients and PDX models that relapsed on Menin-inhibitor treatment. These mutations are distinct from known variants that disrupt Menin's tumor suppressive function in MEN1-syndrome and none have been previously reported¹⁴.

### Resistance Mutations Reduce Drug Binding

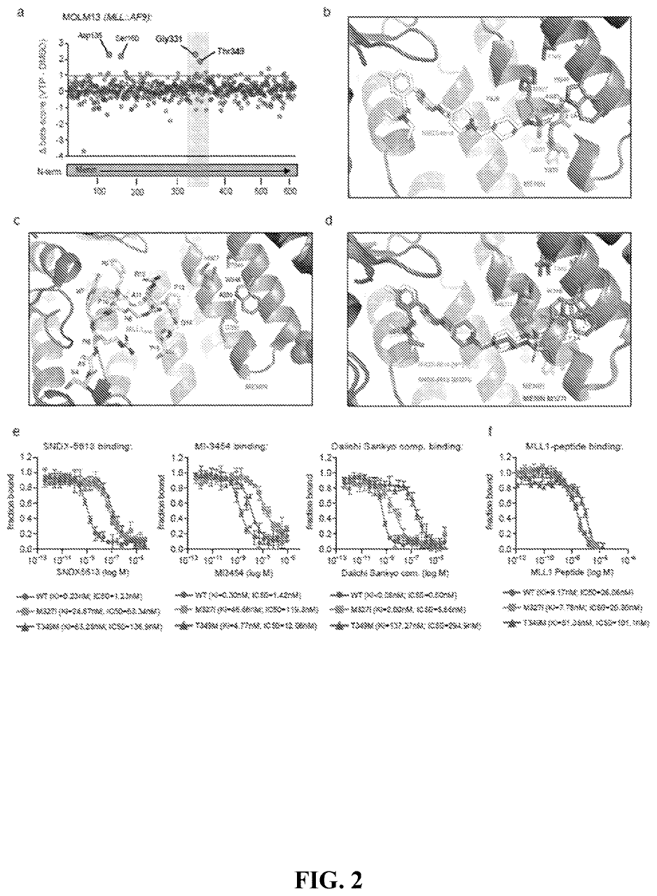

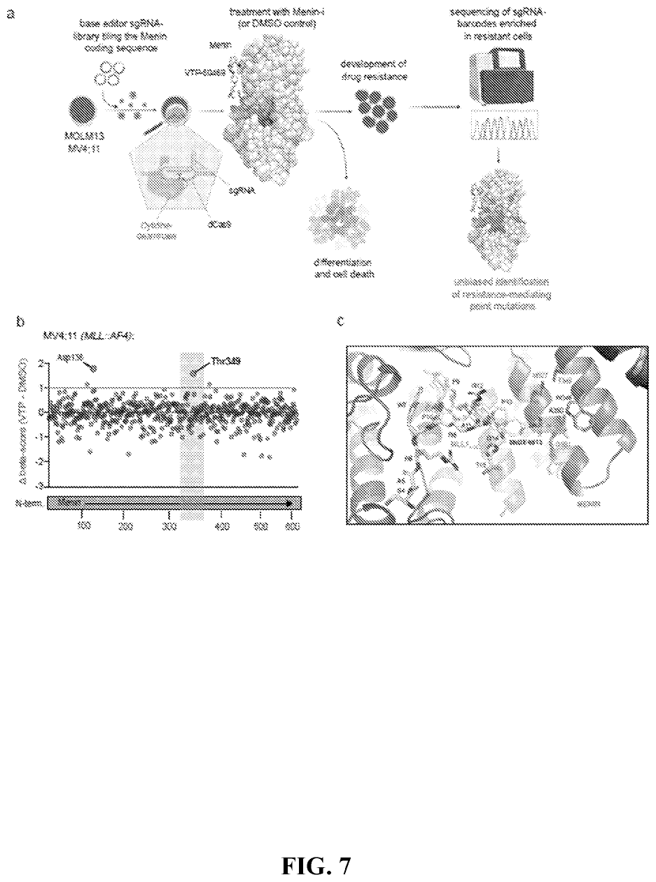

To gain an unbiased view on the spectrum of point mutations that may cause resistance to Menin-inhibitors we conducted a MEN1-focused CRISPR-Cas9 base-editor screen in MOLM13 (MLL::AF9) and MV4;11 (MLL::AF4) cells. A library of 518 single-guide RNAs (sgRNAs) targeting all MEN1-exons and flanking untranslated regions was designed and expressed along with a base-editor system to facilitate C→T base editing in proximity to the sgRNA binding sites¹⁵. Subsequently, cells were exposed to VTP-50469 or DMSO for 12 days and guide barcodes were counted by Next-Generation-Sequencing (NGS) to identify editing sites that confer resistance to drug treatment (FIG. 7A). Using this approach base-editing affecting the majority of amino-acids within Menin could be achieved. Interestingly, the T349-, G331- and 5160 residues were predicted as candidate drivers of Menin-inhibitor resistance in the screen (FIG. 2A, FIG. 7B), but the M327-residue was not base-editable due to the lack of a PAM sequence in this specific region.

The X-ray co-crystal structure of SNDX-5613 bound to Menin revealed that residues M327, G331 and T349 are located in close proximity to the W346 residue (FIG. 2B). There is a strong hydrogen (H)-bond between the sulfonamide oxygen of SNDX-5613 and the indole N—H of W346 that contributes to inhibitor binding. This interaction was previously observed for the related inhibitor, VTP-50469¹³. In addition, we observe a hydrogen bond between the sulfonamide nitrogen of SNDX-5613 and the backbone ketone oxygen of M327. The key amino acids affected by MEN1-mutations (M327, G331, T349) are located around the W346 residue and do not overlap with the binding interface of MLL1 (FIG. 2C, FIG. 7C), indicating they perturb the distal SNDX-5613 sulfonamide interaction. Co-crystallography of SNDX-5613 with the Menin M327I mutant confirmed this effect. As seen in the overlay of the WT and mutant Menin SNDX-5613 structures (FIG. 2D), the branching methyl group of I327 projects toward the cyclohexyl ring of SNDX-5613. The resulting steric clash displaces SNDX-5613, leading to disruption of the H-bonds with W346, as well as with M327, reducing the inhibitor's binding affinity, while leaving the molecule's other interactions with Menin largely unchanged.

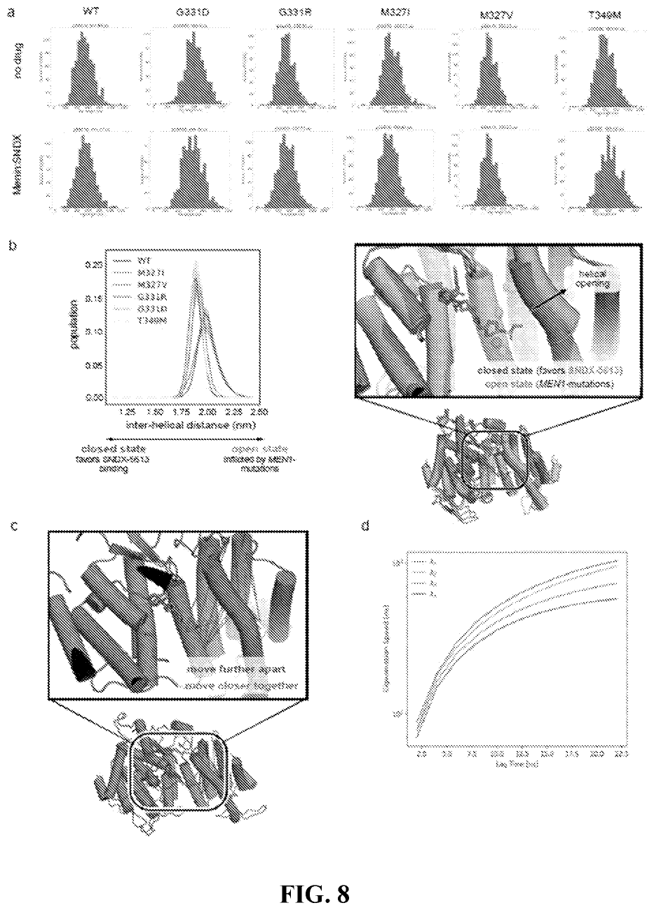

In parallel, we employed computational methods to assess dynamic structural changes in menin incorporating MEN1-T349M, -G331D, -G331R and -M327I/V for the SNDX5613/Menin interface, using the Folding@home distributed computing platform¹⁶. We launched all-atom molecular dynamics simulations of Menin-WT and individual mutant forms in both the presence and absence of SNDX-5613 (FIG. 8A-D) collecting an aggregate 5.469 milliseconds of simulation. Through a combination of Deep Learning methods¹⁷ and Markov State Models¹⁸, we predicted reductions in protein-ligand contacts between Menin and SNDX-5613, perturbing the binding mode and decreasing affinity.

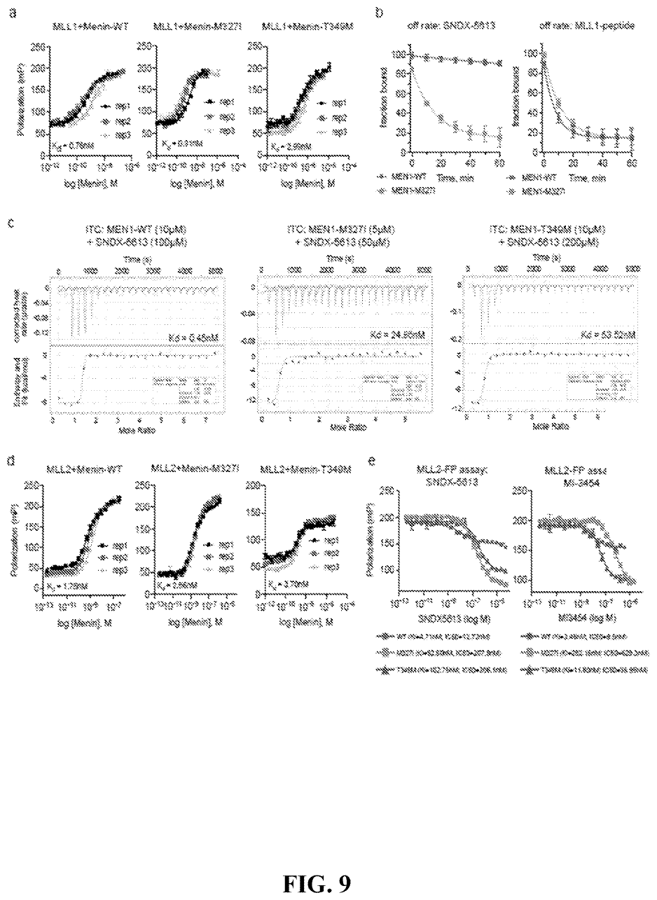

Consistent with the structural and computational results, competitive MLL1 fluorescence polarization (FP) binding assays¹⁹ showed that the M327I and T349M mutants increased the IC50-values of SNDX-5613 for MLL-displacement by 51- and 111-fold compared to WT Menin (FIG. 2E, FIG. 9A-C). The reduced affinity seen in the FP binding assay was consistent with a >30-fold increase in the dissociation off rate of SNDX-5613 from M327I vs WT Menin (FIG. 9B and >50-fold change in mutant binding affinities (Kd) determined by isothermal calorimetry, ITC (FIG. 9C). Similarly, binding of MI-3454, a structural analog of KO-539, and DS-25, a potent compound from the Daiichi-Sankyo series²⁰ were also severely affected by M327I, indicating that Menin-inhibitor resistance at this site exhibit a broad, class effect (FIG. 2E). Interestingly, binding of MI-3454 and DS-25 were differentially affected by the M327I and T349M mutation, respectively (FIG. 2E). Moreover, this mutant effect is not specific to the Menin-MLL1 interaction, since the Menin-inhibitors' ability to block Menin-MLL2 binding was similarly affected by M327I and T349M mutations (FIG. 9D, E). Importantly, the ability of those mutants to bind the MLL1 peptide remained largely intact although a subtle decrease in binding affinity was observed for T349M mutant Menin (FIG. 2F).

Taken together, we independently reproduced the narrow spectrum of MEN1-hotspot mutations observed in patients and PDX by using an unbiased in vitro screening approach. Furthermore, we mapped these residues to an area of the binding pocket that is crucial for stabilizing binding of the Menin-inhibitor molecules but is dispensable for MLL1 binding.

### Mutations Confer Resistance In Vitro

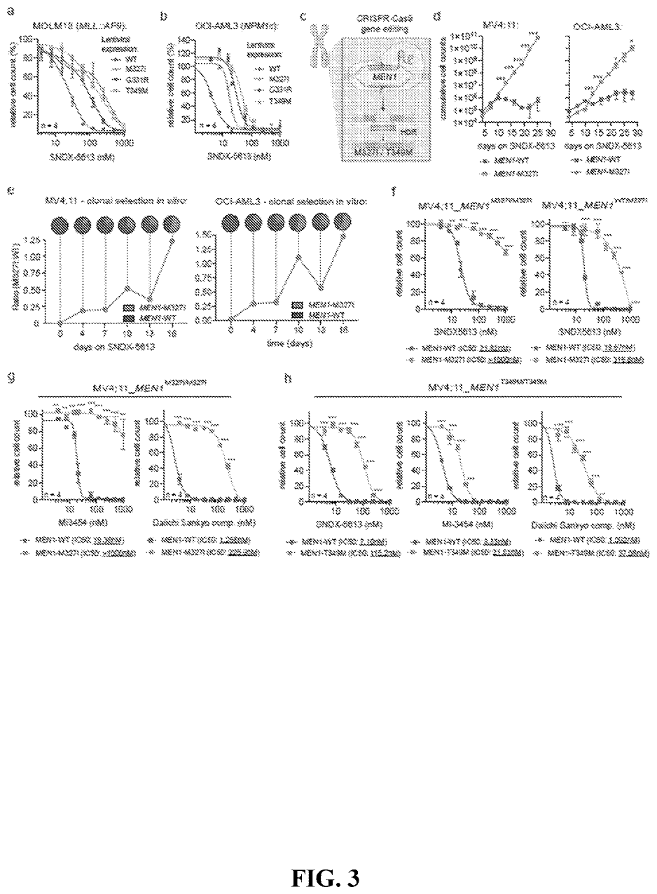

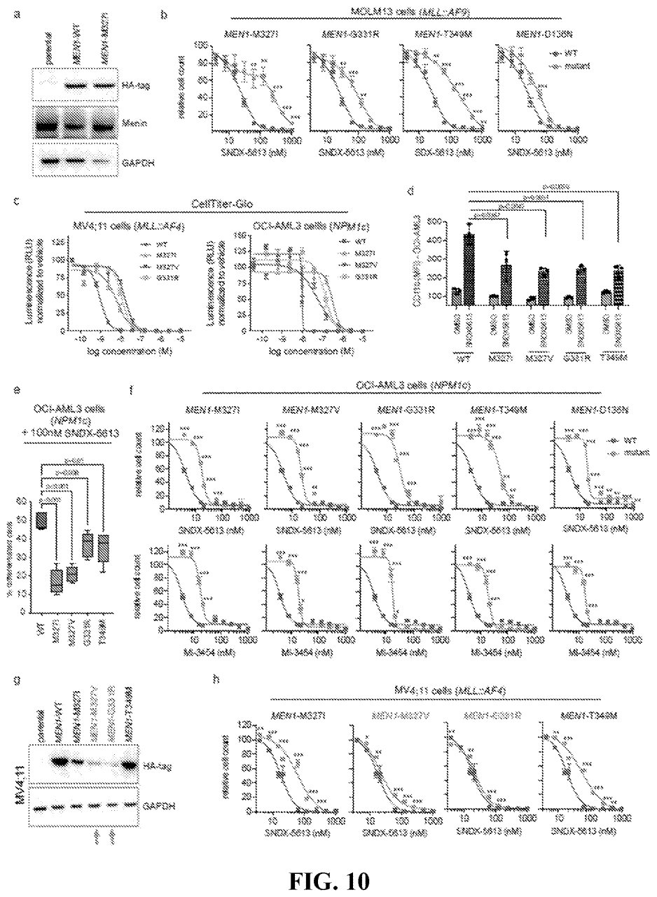

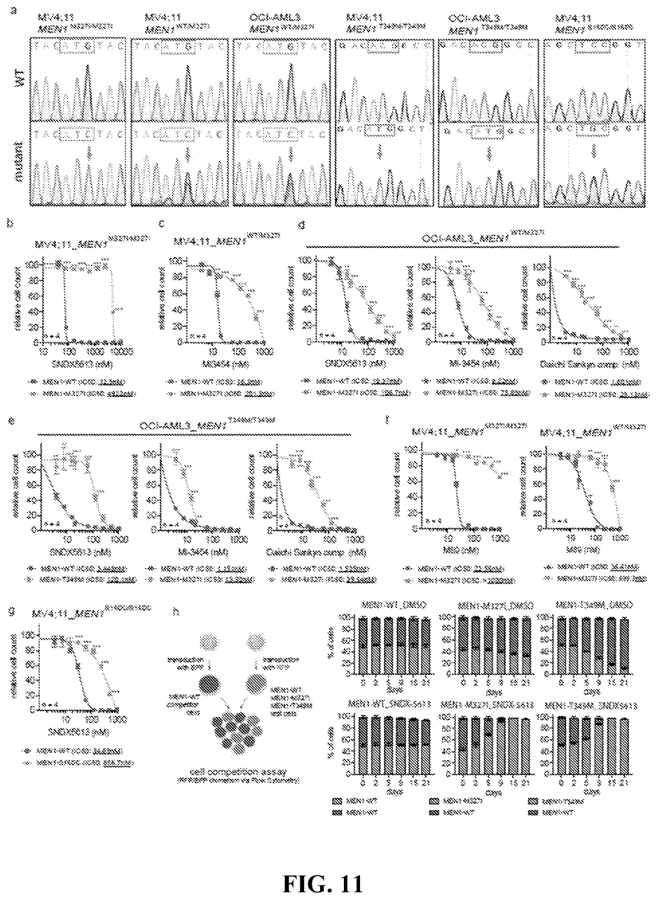

We next assessed whether the mutations conferred resistance to Menin-inhibitor treatment in vitro. We expressed MEN1-M327I, -G331R and -T349M mutants as well as a -WT cDNA in MOLM13 (MLL::AF9), MV4;11 (MLL::AF4) and OCI-AML3 (NPM1c) cells using a lentiviral vector system. Dose-response assays performed by counting the number of viable cells after 10 days of drug treatment demonstrated a robust decrease in SNDX-5613 sensitivity in both KMT2Ar and NPM1c cells expressing these mutations (FIG. 3A, B, FIG. 10A-C, F). Moreover, the induction of myeloid differentiation upon Menin-inhibitor treatment was impaired (FIG. 10D, E). Of note, this resistance phenotype could similarly be observed when cells were treated with the structurally unrelated Menin-inhibitor MI-3454 (FIG. 10F). The degree of drug resistance observed using this system may be dependent on the expression level of each construct (FIG. 10G, H). To overcome the limitations of ectopic expression, we utilized CRISPR-Cas9 in conjunction with a homology directed repair template to edit M327I or T349M mutations into the endogenous MEN1 coding sequence of MV4;11 and OCI-AML3 cells (FIG. 3C). After nucleofection, M327I-edited or unedited bulk populations of cells were exposed to 50 nM of SNDX-5613 and cell counts were monitored over the course of a month (FIG. 3D). Unedited MV4;11 and OCI-AML3 cells stopped dividing shortly after exposure to the Menin-inhibitor, while CRISPR-Cas9-edited cells continued growing beyond 28 days which was accompanied by positive selection for the M327I mutation (FIG. 3E), providing in vitro support for the concept of inhibitor-driven clonal selection by this mutation. After screening of single cell clones, we established cell lines harboring M327I and T349M mutations (FIG. 11A). MEN1^{M327I/M327I} MV4;11 cells did not respond to SNDX-5613 at physiologically relevant doses with IC₅₀ values shifting from the low nanomolar range to over 1 μM when the mutation was present (FIG. 3F, FIG. 11B). MEN1^{M327I/WT} cell lines retained some sensitivity to Menin-inhibition, however IC₅₀ values were increased by 16-fold compared to MEN1-wild-type cells (FIG. 3F). Consistent with the findings from our binding assays, we observed reduced sensitivity to SNDX-5613, MI-3454 and the Daiichi-Sankyo compounds in M327I- and T349M-mutant MV4;11 and OCI-AML3 cells (FIG. 3G, H, FIG. 11C-E). Of note, the observation that MI-3454 binding in the fluorescence polarization assay was less affected by T349M was reflected in the dose-response curves (FIG. 3H, FIG. 11E). We further found that cells expressing MEN1-M327I were also resistant to another Menin inhibitor. M-89, and we confirmed that the more N-terminal located S160C mutation conferred a similar degree of SNDX-5613 resistance (FIG. 11F, G). To answer the question of whether the presence of recurrent MEN1 mutations may impact fitness of leukemia cells in the absence of a Menin-inhibitor we performed a cell competition assay (FIG. 11H). SNDX-5613 exposure led to a rapid selection of M327I and T349M mutant cells while the chimerism between mutant and non-mutant cells decreased under DMSO treatment (FIG. 11H). This effect was particularly pronounced for T349M mutant cells and only subtle when M327I was present, corresponding to the MLL1-binding assay (FIG. 2F). In summary, we could demonstrate that endogenous or exogenous expression of recurrent MEN1 mutants in KMT2Ar or NPM1 mutant AML cell lines is sufficient to induce drug resistance to a panel of currently available Menin-inhibitors.

### Mutations Prevent Menin Inhibitor-Induced Chromatin and Gene Expression Changes

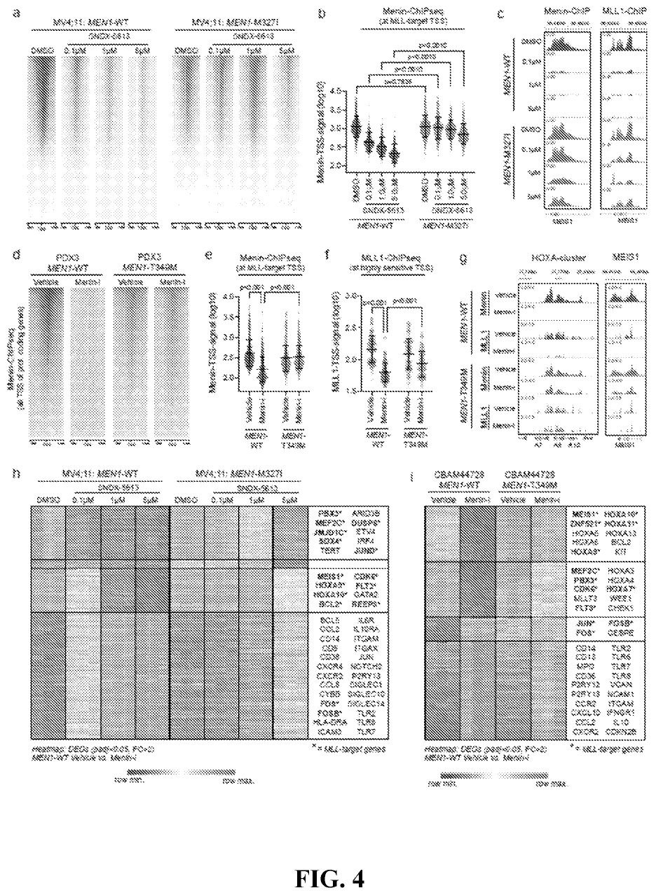

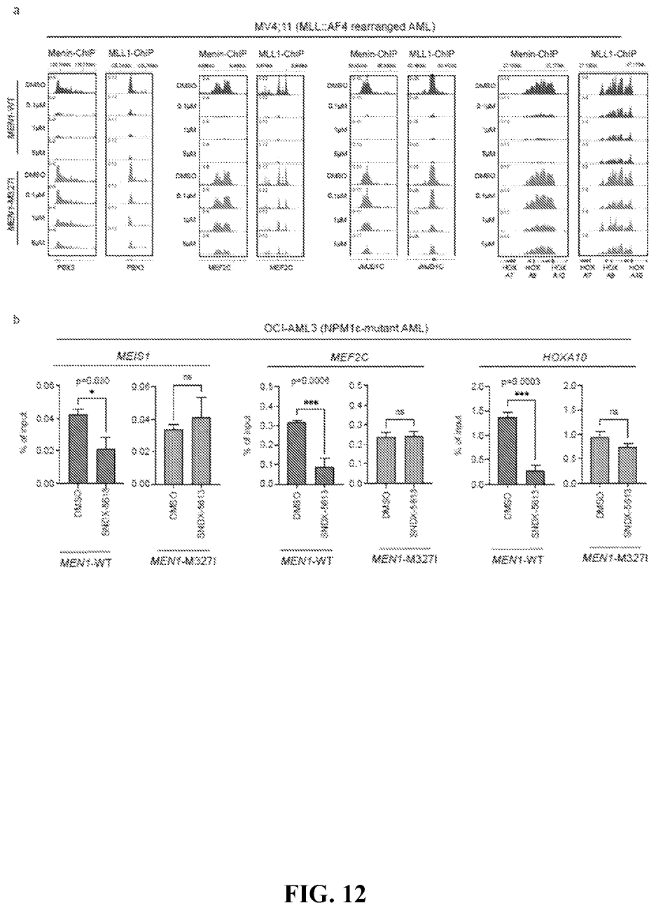

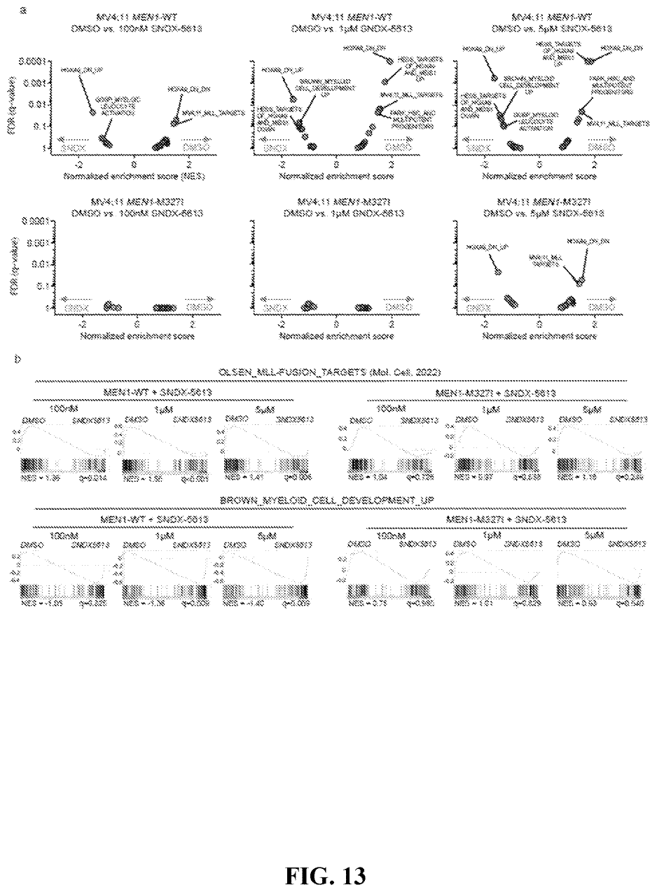

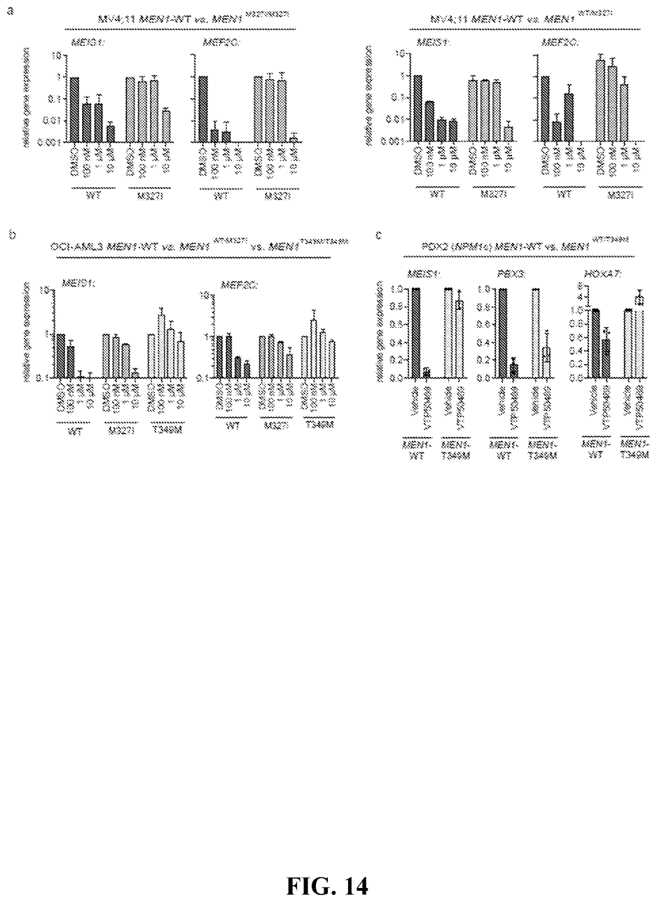

We next investigated whether mutations that confer Menin inhibitor resistance attenuate the changes in chromatin and gene expression seen with inhibition of the Menin and MLL1 interaction in MEN1-WT leukemia cells. Chromatin Immunoprecipitation Sequencing (ChIP-Seq) was performed after treatment of the MV4;11 MEN^{M327I/M327I} and WT control cell lines with SNDX-5613 to assess genome-wide chromatin occupancy of Menin and MLL1 (FIG. 4A). Treatment of MEN1-WT cells led to near-complete and global displacement of Menin from chromatin with exposure to as little as 100 nM of SNDX-5613. In contrast, the M327I mutant cells retained Menin on chromatin and only showed a partial decrease in Menin chromatin occupancy even when exposed to 5 μM of SNDX-5613 (FIG. 4A, B). Consequently, inhibitor induced MLL-eviction from key target loci, including MEIS1, was largely abrogated in M327I mutant MV4;11 cells (FIG. 4C, FIG. 12A). Consistent with these findings in the MLL::AF4-rearranged MV4;11 cells, displacement of Menin from chromatin was blunted by both M327I and T349M in NPM1c-mutant OCI-AML3 cells (FIG. 12B). Similarly, treatment of mice that were engrafted with the T349M-mutant PDX3 failed to displace Menin from chromatin after oral Menin-inhibitor treatment in vivo (FIG. 4D, E). The profound loss of MLL1 from promoters with the highest degree of Menin displacement (>80%) was blunted in T349M mutant cells (FIG. 4F, G). To delineate the consequences of the altered chromatin binding dynamics of Menin and MLL1 under SNDX-5613 treatment, we performed RNA-sequencing in MEN1-mutant MV4;11 cells and PDX3 samples. In line with our observations on chromatin, MEN1-M327I mutant MV4;11 cells showed a high degree of resistance to SNDX-5613-mediated changes in gene expression (FIG. 4H, I). Specifically, the repression of canonical Menin-MLL1 target genes as well as the induction of gene programs that are associated with myeloid differentiation were largely abrogated in M327I-mutant cells and only a partial response could be observed under treatment with 5 μM of SNDX-5613 (FIG. 4H, FIG. 13A, B). Of note, these effects on gene expression were consistent between the KMT2Ar MV4;11 and NPM1c-mutant OCI-AML3 cells (FIG. 14A, B). Likewise, treatment of mice engrafted with T349M-mutant PDX3 failed to sufficiently repress Menin-MLL target genes or induce differentiation-associated gene signatures in human leukemia cells as compared to mice engrafted with isogenic MEN1-WT cells in response to Menin-inhibition (FIG. 4I). Quantitative real-time PCR analysis in leukemia cells from the NPM1c-mutant PDX2 (MEN1-T349M vs. WT) showed the same phenomenon (FIG. 14C). Overall, we demonstrate in both KMT2Ar and NPM1c model systems that MEN1 resistance mutations prevent Menin-inhibitor induced displacement of the Menin-MLL1 protein complex from chromatin at critical target genes, which consequently abrogate gene expression changes that are required to terminate leukemic self-renewal and induce myeloid differentiation.

### Conclusion

We report that somatic mutations in MEN1 that confer resistance to Menin inhibitor treatment are frequently acquired in KMT2Ar and NPM1c leukemia cells in patients and in preclinical PDX models of disease. These mutations lead to recurrent changes in amino acid residues M327, G331, T349 and S160 and induce drug resistance to structurally distinct classes of Menin-inhibitors, some of which, including SNDX-5613, recently entered early-phase clinical trials. Remarkably, amino acids G331, T349 and S160 were also identified as putative targets for resistance-development in a base-editor screen of the MEN1 gene, strengthening the confidence in the predictive value of those experimental systems. These new MEN1-mutations decrease the affinity of the Menin/inhibitor interaction, preventing drug-induced displacement of the Menin-MLL1-complex from chromatin and thereby abrogating critical gene expression changes. The affected amino acids are essential for small molecule binding, but not for MLL1 association with Menin which allows for continued oncogenic activity of the MLL1-Menin complex on chromatin. Most importantly, the discovery of acquired mutations in Menin validates the Menin/MLL1 interaction as a key oncogenic driver in patients with AML harboring KMT2A rearrangements or NPM1c mutations and, as such, represents a promising therapeutic target. While not wishing to be bound by theory, the observation that these mutations confer resistance across multiple Menin inhibitors indicates that next-generation inhibitors can be designed to circumvent this mechanism of resistance. Structure-guided drug design to derive second-generation compounds that effectively block MLL1 binding while avoiding interactions with the residues affected by acquired MEN1 mutations, can be a strategy to overcome acquired resistance to first generation Menin-inhibitors. Given these findings, prospective monitoring of MEN1 mutation status, using diagnostic tests as described herein, can be considered in patients receiving Menin-inhibitor therapy, for example, for patients with delayed or incomplete responses, to inform treatment decision-making. This is the first report of clinically occurring somatic mutations that mediate acquired resistance to small molecules targeting chromatin-binding protein complexes. These data support chromatin complexes and epigenetic mechanisms as therapeutic targets in cancer but also show that a common mechanism of resistance to therapies targeting these complexes may result from acquired mutagenesis of essential, non-driver epigenetic regulators.

### Methods

### Patient Subjects and Primary Sample Acquisition

The human subjects that are part of this report were enrolled at Memorial Sloan Kettering Cancer Center (MSKCC) or Dana-Farber Cancer Institute (DFCI) in the AUGMENT-101 trial, a Phase 1/2, open-label, dose-escalation and dose-expansion cohort study of SNDX-5613 in patients with relapsed/refractory leukemias, including those harboring an MLL/KMT2A gene rearrangement or Nucleophosmin (NPM1) mutation. Additional patient specimens were obtained from the centralized sample bank of Syndax pharmaceuticals. Patients provided oral and written consent to participate in the clinical trial and the sample banking and sequencing program at MSKCC or DFCI (IRB 19-448 and 06-107). Assessments of clinical response with bone marrow biopsies and peripheral blood assessment were performed monthly.

### Patient-Derived Xenografts (PDX)

For the assessment of long-term responses and the characterization of drug-resistance in the xenograft model system, we used 6 different PDX models of AML (5×KMT2Ar, 1×NPM1c-mutant), that were previously established and characterized in the “Center for Pediatric Cancer Therapeutics” (CPCT) at Dana-Farber Cancer Institute (DFCI). In vivo experiments were performed at DFCI under the “Institutional Animal Care and Use Committee” (IACUC) protocol: #16-021. NOG-mice (Taconic. 10-14 weeks of age) were injected with 250.000 cryo-preserved leukemia cells of each graft without prior conditioning and engraftment was monitored in the peripheral blood by detecting the chimerism between human CD45 (PE; clone: HI30; Biolegend) and mouse CD45 (APC-Cy7; clone: 30-F11; Biolegend) using flow cytometry every 3-4 weeks (LSRFortessa™; BD Biosciences). When human cells were consistently detectable in all mice of a given graft, oral treatment with VTP-50469 was initiated (0.03% drug supplemented rodent diet; drug supplied by Syndax Pharmaceuticals; diet produced by ENVIGO). Oral treatment was given for 4 weeks or until no leukemia cells were detectable anymore in the peripheral blood of mice. After discontinuation, mice were closely monitored by peripheral blood chimerism, and treatment was re-initiated upon relapse. Animals that reached the study endpoint were euthanized using CO₂ inhalation and subsequent cervical dislocation. Tibias, femurs, iliac crests, and lumbar vertebrates were cleaned using TX329 cotton wipes (Texwipe) and crushed using mortar and pestle to extract bone marrow cells. Spleen cells were extracted by straining the organ through a 40 μM nylon cell filter (FALCON). The human leukemia cell burden in blood, spleen and bone marrow was measured by detecting the chimerism between human CD45 (PE; clone: HI30; Biolegend) and mouse CD45 (APC-Cy7; clone: 30-F11; Biolegend) and the differentiation status of human cells was assessed using anti-CD13 (PerCp-Cy5.5; clone: WM-15; Biolegend), anti-CD14 (PE-Cy7: clone: M5E3; Biolegend) and anti-CD11b (FITC: clone: IRCF44; Biolegend).

### Targeted DNA-Sequencing of Patient and PDX Material

Diagnostic bone marrow aspirates were collected from patients before enrollment on the trial and at the time of relapse. Mononuclear cells were isolated via Ficoll gradient centrifugation and genomic DNA was extracted before CLIA-approved targeted sequencing was performed (MSKCC-IMPACT). Bone marrow from PDX (at relapse on or after VTP-50469 treatment or treatment naive as control) was extracted via crushing of the tibias, femurs, iliac crest, and lumbar vertebrates using mortar and pestle. Subsequently, murine cells were removed using the “Mouse Cell Depletion Kit” (Miltenyi Biotec) and genomic DNA was isolated from human cells using the DNeasy Blood and Tissue Kit™ (QIAGEN). Samples were subjected to targeted sequencing for oncogenic driver mutations using the MSK-IMPACT pipeline as previously described²¹.

### Nomenclature of MEN1 Mutations

All annotations of amino-acid changes inflicted by MEN1 mutations reported in this manuscript are based on the Menin-Isoform #1 (RefSeq: NM_000244.3).

### Experimental Procedure for the Generation of the Menin:SNDX-5613 Structures

Menin crystals were grown at 21° C. using the sitting drop vapor diffusion method. Purified Menin at 11 mg/ml in 10 mM Tris pH 7.5, 50 mM NaCl, 1 mM TCEP was pre-incubated with inhibitor compounds dissolved in DMSO. The pre-incubation was done at 0.6 mM inhibitor concentration. For crystallization. 1.0 μL of the protein inhibitor complex was mixed 0.5 μL of seeds and 1.5 μL of a reservoir solution. SNDX-5613 was co-crystallized using as reservoir condition 0.1 M HEPES pH 7.9, 24% PEG 3350, 0.2 M Magnesium Nitrate and 20% Ethylene Glycol. Crystals were flash cooled in cryo protection solution. For data acquisition, the crystal temperature was kept at 100 K. Diffraction data were collected at the Australian Synchrotron (beamline MX2) using a 16M pixel Dectris Eiger detector. Raw diffraction data were processed and scaled using XDS software. The structures were solved by molecular replacement with MOLREP in CCP4i using as search model the coordinates previously solved structures of menin (PDB ID 6PKC). The program REFMAC5 was used for full structure refinement. The refined coordinates of the complex structures have been deposited in the RCSB Protein Data Bank.

Synthesis and Cloning of MEN1 Mutants into Lentiviral Constructs

The Menin-coding sequence was codon optimized and gene synthesis was performed via Twist Bioscience into a bacterial vector system (pTWIST-Amp). Small double-stranded DNA-blocks (gblocks, Integrated DNA Technologies) harboring the MEN1-M327I, M327V, G331R, T349M or D136N mutations were synthesized and cloned into pTWIST-Amp-MEN1 using a restriction enzyme mediated digest (BamHI+HindIII→M327I, M327V, G331R, HindIII+AfeI→T349M, SpeI+ClaI→D136N) and T4-ligase based cloning approach (all enzymes: New England BioLabs) in order to insert the mutations into the MEN1-coding sequence. The codons used were: M327I: ATG→ATC; M327V: ATG→GTG; G331R: GGT→CGT; T349M: ACC→ATG; D136N: GAC→AAC. Subsequently, MEN1 was amplified from the generated pTWIST template vectors using the Q5 High-Fidelity 2× Master Mix™ (New England BioLabs) and primers (forward: 5′-CCCAGGGGCTAGCATGGGTTTGAAAGCGGCGCAGA-3′; reverse: 5′-AGAGGTTGATTGTCGACTTAACGCGTTTATGCATAGTCCGGGACATCATACGGAT AGCCGGCGTAGTCGGGCACGTCGTAGGGGTAAAGTCCCTTCCTTTGTCGTTTCAG AA-3′) under addition of a double-HA-tag to the C-terminus of Menin. The PCR product was gel-purified using the QIAquick Gel Extraction Kit™ (QIAGEN) and cloned into the pLEX-puro lentiviral vector system using T4-ligase after digest with NheI and MluI (New England BioLabs).

### Purification of Recombinant Menin

Wild-type human Menin was codon optimized for E. coli expression, synthesized, and cloned in pET28+ derived vectors (Twist Biosciences) and the resulting vector was further subcloned with a synthesized gBlock (IDT) to produce a N-terminal StrepII-Avi-TEV Menin fusion. Mutant protein plasmids were generated by site-directed mutagenesis using the Q5® Site-Directed Mutagenesis Kit (NEB) following manufacturer instructions, all resulting plasmids were sequence verified. Recombinant proteins were expressed as N-terminal StrepII-Avi-TEV fusions in BL21-DE3 Rosetta cells following standard protocols. Briefly, each expression was performed at 4 L scale in LB media. 10 mL of overnight starter cultures was added to each liter of LB at 37° C., and protein expression induced by addition of 1 mM IPTG at OD600 of 0.6 following change of temperature to 18° C. overnight. The overnight expression cultures were harvested by centrifugation at 4000 g for 20 min and resuspended in buffer containing 50 mM tris(hydroxymethyl)aminomethane hydrochloride (Tris-HCl) pH 8.0, 200 mM NaCl, 2 mM tris(2-carboxyethyl)phosphine (TCEP), 1 mM phenylmethylsulfonyl fluoride (PMSF), 10 μM Bestatin, 2 μM E-64, 1 μM Pepstatin and 10 μM Leupeptin, and lysed by sonication. Following ultracentrifugation, the soluble fraction was passed over Strep-Tactin XT (IBA) affinity resin and eluted with wash buffer (50 mM Tris-HCl pH 8.0, 200 mM NaCl, 2 mM TCEP) supplemented with 50 mM biotin (MCE). The affinity-purified protein was subject to ion exchange chromatography (Poros 50HQ) followed by size exclusion chromatography (Superdex 200 10/300 GL) in 50 mM HEPES pH 7.4, 200 mM NaCl and 2 mM TCEP. The protein-containing fractions were concentrated using ultrafiltration (Millipore) and flash frozen in liquid nitrogen at 10 μM concentration.

### Crystallography Methods

### Menin (WT)-SNDX-5613

Menin protein at 11 mg/ml in 10 mM Tris pH 7.5, 50 mM NaCl, 1 mM TCEP was used for setting up crystallization. Menin-5613 crystal was obtained using cross-seeding from other Menin co-crystals. For Menin-SNDX-5613 crystallization, 1.0 μL of the protein inhibitor complex was mixed 0.5 μL of seeds and 1.5 μL of a reservoir solution using the sitting drop vapor diffusion method. The reservoir solution contains 0.1 M HEPES pH 7.9, 24% PEG 3350, 0.2 M Magnesium Nitrate and 20% Ethylene Glycol. Crystals were obtained within 10 days of incubation at 21° C.

### Menin (M327I)-SNDX-50613

Menin (M327I) protein at 10.7 mg/ml in 10 mM Tris pH 7.5, 50 mM NaCl, 1 mM TCEP was used for setting up crystallization. Menin (M327I)-SNDX-50613 crystals were obtained using sitting drop vapor diffusion method with 1 ul of protein-inhibitor mix and 1 ul of reservoir buffer. The reservoir buffer contains 0.1 M MES pH 6.2, 16% PEG 3350, 0.2 M Potassium thiocyanate and 20% Ethylene Glycol. Crystals were obtained within 7 days of incubation at 21° C.

While most annotations of Menin mutations are based on isoform 2, which encodes 610 amino acid residues, some annotations are based on an alternate transcript with 615 amino acids. Biancaniello et al.²² stated that the 615-amino acid isoform be used as the standard reference for Menin, which was the reference used to report the crystallography data. References made to the 610-residue isoform in some instances will result in a 5-residue shift in register.

### Fluorescence Polarization

The following peptides were synthesized by GenScript: N-terminal FITC-conjugated MLL1₄₋₄₃(C-A) peptide probe (SARWRFPARPGTTGGGGGGGRRGLGGAPRQRVPALLLPPGY) with a C-A modification that improves binding to Menin¹⁹, MLL1₄₋₄₃(C-A) (SARWRFPARPGTTGGGGGGGRRGLGGAPRQRVPALLLPPGY) and MLL1₄₋₄₃(WT) (SCRWRFPARPGTTGGGGGGGRRGLGGAPRQRVPALLLPPGY). N-terminally FITC-labelled and unmodified MLL2 peptides (MLL2(15-48) SARGRFPGRPRGAGGGGGRGGRGNGAERVRVALR) were synthesized at Genscript. To derive the FITC-MLL1₄₋₄₃(C-A) peptide K_{d}. FITC-conjugated MLL1₄₋₄₃(C-A) peptide probe at final concentration of 1 nM was mixed with increasing concentration of purified StrepII-Avi-TEV-Menin (5 μM final top concentration, 2-fold, 23-point dilution and a buffer control) in an assay buffer (50 mM Tris pH 7.5, 200 mM NaCl, 0.1% Pluronic F-68 solution (Sigma)) in 384-well microplates at 15 μL assay volume (Corning, 4514) and incubated for 30 min at room temperature (RT). For all assays change in fluorescence polarization was monitored using a PHERAstar FS microplate reader (BMG Labtech). The FITC-conjugated MLL1₄₋₄₃(C-A) peptide K_{d} was obtained from a fit in GraphPad Prism 9 using one site total model and averaged from three independent runs with three replicates (n=3, FIG. 8). In competitive titration experiments Menin proteins (1 nM WT, 1 nM M327I or 3 nM for T349M) and 1 nM FITC-conjugated MLL1₄₋₄₃(C-A) peptide probe were incubated in an assay buffer for 30 min. SNDX-5613 was then dispensed to 384-well microplate (Corning, 4514) containing 15 μL of the assay mix using D300e Digital Dispenser (HP) and normalized to 1% DMSO followed by 90 min incubation. The fluorescence polarization was monitored by PHERAstar FS microplate reader (BMG Labtech). The MLL1₄₋₄₃(WT) and MLL1₄₋₄₃(C-A) peptide titrations were performed by addition of 7.5 μL of 2-fold 23-point serial dilution into 7.5 μL of assay mix with the final concentrations of 1 nM FITC-conjugated MLL₄₋₄₃ peptide probe, 1 nM WT Menin or 1 nM Menin M327I or 3 nM Menin T349M in assay buffer. The fluorescence polarization was monitored by PHERAstar FS microplate reader (BMG Labtech). For analysis of the competitive titration experiments the last 10 cycles of the data were averaged to obtain technical replicates. Data from three independent replicates (n=3) was plotted and IC₅₀ values estimated using variable slope equation in GraphPad Prism 9. The calculation of Kᵢ values was performed using Kᵢ calculator available at websites.umich.edu/˜shaomengwanglab/software/calc_ki/index.html²³. The assay parameters used for calculation of Kᵢ were: 1 nM probe for all assays, 1 nM WT Menin, 1 nM Menin M327I, 3 nM Menin T349M, with K_{d} (mean±S.D., N=3) of 0.76±0.77 nM for WT Menin, 0.91±0.99 nM for Menin M327I, 2.99±1.62 nM for Menin T349M.

### Fluorescence-Based Kinetic Binding Assays

Off-rates for test compounds were determined using jump dilution experiments in the HTRF assay measuring FITC-MLL4-43 binding to HIS-Menin. Briefly, test compounds (30 nM) were pre-incubated for 1 hr with 30 nM HIS-Menin prebound to 30 nM Lanthascreen anti-HIS-Tb antibodies in 10 μL of menin assay buffer (50 mM Tris, pH 7.4, 50 mM NaCl, 5 mM DTT, 0.01% TX100) containing 0.02% fatty acid free BSA. After pre-incubation, 1 μL of the assay mixture was transferred to 100 μL of menin assay buffer containing 30 nM FITC-MLL-4-43 (1000×Ki) in white, opaque 384-well plates. The HTRF signal was measured at various times using a 320 nm excitation and 520 and 620 nm emission wavelengths with a 50 us delay and a 200 us window with the gain set to 2100 using a BMGlabTech ClarioStar Plus plate reader. The data was normalized to % inhibition at each time point, where 0% is the HTRF signal in the presence of 0.3 nM HIS-menin without test compound, and 100% is the HTRF signal in the absence of HIS-menin. The resulting % inhibition values were fit to a one-site exponential dissociation model using XlFit to determine the dissociation rate.

### Isothermal Titration Calorimetry

All calorimetric experiments were carried out in ITC buffer (25 mM Hepes (pH 7.5), 200 mM NaCl, 1.0 mM TCEP) at 25° C. using an Affinity ITC from TA Instruments (New Castle, DE) equipped with autosampler. SNDX-5613 was dissolved in DMSO and diluted with the ITC buffer to final concentrations (50-200 μM. 1% DMSO). Protein sample was adjusted to contain 1% DMSO final concentration. The calorimetric cell, containing buffer or Menin (10 μM WT Menin, 5 μM M327I Menin, or 10 μM T349M Menin) was titrated by injecting 2.5 μl of SNDX-5613 solution with the concentration of 100 μM, 50 μM or 200 μM, respectively, 24 times with stirring speed at 75 rpm. The resulting isotherms were subtracted against buffer runs and fitted with a single-site model to yield thermodynamic parameters of ΔH and ΔS, stoichiometry, and KD using NanoAnalyze software (TA Instruments).

### Cell Culture and Lentiviral Transductions

Cell lines were obtained from the “American Type Culture Collection” (ATCC) or the “Deutsche Sammlung von Mikroorganismen und Zellkulturen” (DSMZ) and cultured in RPMI-1640™ (for MOLM13, MV4;11 and OCI-AML3) or DMEM™ (for HEK-293T)+10% fetal bovine serum (FBS)+1% penicillin/streptomycin (P/S) (Life Technologies/Thermo Fisher Scientific) at 37° C., 5% CO2 atmosphere and 95% humidity. Cell lines were tested and maintained mycoplasma negative throughout all experiments. For the production of lentivirus HEK-293T cells were seeded in 10 cm tissue culture treated dishes (Corning) 24 h before transfection to achieve 80% confluence. pLEX-puro plasmids (5 μg) containing a MEN1-WT, -M327I, -M327V, -G331R, -T349M, -D136N or the MEN1-CRISPR-Cas9-base-editor library were transfected along with lentiviral packaging plasmids (2 μg pMD2G+5 μg psPAX2) using 30 μl XtremeGene9™ DNA transfection reagent (Sigma-Millipore) in 1.8 ml Opti-MEM per 10 cm dish containing 4 ml DMEM+10% FBS (Life Technologies/Thermo Fisher Scientific). Medium was exchanged with 10 ml fresh DMEM+10% FBS and viral supernatants were harvested 24 h later by filtering through a 0.45 μm Nalgene syringe filter (Thermo Fisher Scientific). Viral supernatants were frozen at −80° C. or directly used to infect target cells. For lentiviral transduction target cells were resuspended in crude viral supernatants and spin-infection was done in 50 ml Falcon-tubes for 2 h at 2000 rpm in a centrifuge heated to 37° C. Subsequently, viral supernatants were removed, and cells were resuspended in RPMI-1640™+10% FBS+1% P/S. Puromycin was added to pLEX-puro transduced cell lines at a final concentration of 1 μg/ml 48 h after transduction to select for cells expressing the respective constructs.

### CRISPR-Cas9 Base-Editor Screening

An sgRNA-library of 518 single-guide RNAs (sgRNAs) targeting all MEN1-exons and flanking untranslated regions was designed, synthesized, and cloned into the pRDA_256 vector system at the Broad Institute of Harvard and MIT via the Genetic Perturbation Platform (GPP). Production of lentiviral particles and viral transduction was performed as described herein. MOLM13 and MV4;11 cells were transduced with the base-editor library and selected with Puromycin 1 μg/ml, Thermo Fischer Scientific) for 6 days. After selection, a baseline sample (5×10⁶ cells) was harvested for DNA-extraction and the remaining cells were split into 10 separate non-tissue culture treated T75 culture flasks. Each 5 of these flasks were treated with 50 nM of VTP-50469 or DMSO as control for 12 days and split every 3 days during this time. At the end of the experiment, cells were harvested and DNA was extracted using the DNeasy Blood and Tissue Kit™ (QIAGEN). Library construction, Next-Generation sequencing, and generation of an sgRNA count matrix were performed via the GPP at the Broad Institute. Beta-scores were calculated for each condition (baseline vs. endpoint) using the MaGECK-MLE pipeline. Differential beta scores for each guide-RNA were calculated by subtracting the control beta-score from the VTP-50469 values.

### CRISPR-Cas9-Based Gene-Editing of MEN1 by Homology Directed Repair (HDR)

MV4;11 and OCI-AML3 cell lines were passaged 24 h prior to nucleofection. The guide RNA (150 μmol) (sequence: 5′-CATCTACCCCTACATGTACC-3′) was added to Alt-R S.p. HiFi Cas9 nuclease (Integrated DNA Technologies) (125 μmol) followed by an incubation at room temperature (RT) for 20 min to allow formation of ribonucleoprotein (RNP) complexes. 2×10⁵ cells were sedimented at 200 g for 10 min at RT, washed with PBS and resuspended in the SG Cell Line Nucleofector™ Solution (Lonza). Cells, Alt-R™ Cas9 Electroporation Enhancer (Lonza) (120 μmol) and the HDR donor oligonucleotide containing the MEN1-M327I mutation as well as a silent mutation at the protospacer adjacent motif (PAM) site (120 μmol) (sequence:5′-TCCCGCACATTGCGGTTGCGACAGTGGTAGCCAGCGAGGTAGATGTAGGGGTAG ATGTGTTCATCCCGATAGTAGGTCTTGGCT-3′) were added to the RNP complex. For wildtype (WT) control, phosphate buffered saline (PBS) (Gibco™, Fisher Scientific) was added to the resuspended cells. Nucleofection was performed using a 16-well 20 μl Nucleocuvette™ stripe (Lonza) in a 4D-Nucleofector™ X Unit (Lonza) (program FF-100). Cells were incubated overnight in RPMI-1640+10% FCS+1% P/S containing 1 μM Alt-R™ HDR enhancer V2 (Lonza) (or DMSO for WT control). The following day, the medium was replaced by RPMI-1640+10% FBS+1% P/S. Single clones were selected in methylcellulose (MethoCult™ M3234, Stemcell™ Technologies) supplemented with 10% RPMI-1640, 10% FBS, 1% P/S and 25 nM SNDX-5613 (or DMSO for WT control), starting 3 days after nucleofection, and expanded in RPMI-1640+10% FCS+1% P/S supplemented with 25 nM SNDX-5613 (or DMSO for WT control). For the in vitro clonal selection assay of bulk nucleofected cells over time, 5×10³ cells (3 replicates per condition) were seeded in RPMI-1640+10% FBS+1% P/S supplemented with 50 nM SNDX-5613 (or DMSO for WT control) 3 days after nucleofection. Cells were replated in fresh medium+drug every 3 days and viable cells were counted using the LSRFortessa™ (BD Biosciences) flow cytometer. Cumulative cell growth was calculated over 28 days. At each timepoint, cell pellets were frozen and genomic DNA was extracted subsequently using the DNeasy Blood and Tissue Kit™ (QIAGEN). Digital droplet-based PCR was performed from the genomic DNA to assess editing efficiency in the bulk cells over time. To analyze HDR efficiency in single cell clones, genomic DNA was PCR amplified using the Q5 High-Fidelity 2× Master Mix™ (New England BioLabs) and primers (forward: 5′-CCCTCAGCCCTGCCTTTTCTGC-3′; reverse: 5′-AGTCCTGGACGAGGGTGGTTGG-3′), and the resulting 641 bp fragment containing the Cas9 cut site was gel-purified using the QIAquick Gel Extraction Kit™ (QIAGEN). Sanger sequencing was used to analyze HDR efficiency (primer 5′-CTGGGATCTTCCTGTGGCCCCT-3′).

### Assessment of Proliferation In Vitro

To determine Menin-inhibitor responses of MOLM13, MV4;11 or OCI-AML3 cell lines expressing MEN1 mutants, 3×10³ cells were plated per well in a non-tissue culture treated 96-well plate. The Menin-inhibitors were titrated on these cells in the following dose-range: DMSO, 3.9 nM, 7.813 nM, 15.625 nM, 21.25 nM, 62.5 nM, 125 nM, 250 nM, 500 nM, 1000 nM. Cells were treated in triplicates on each plate and the experiment was repeated 4 times. After 6 days, cells were split 1:7 and counts of viable cells in each well were determined after 10 d of treatment by flow cytometry (LSRFortessa™ with High-throughput sampler (HTS), BD Biosciences). Relative cell counts were calculated as % of DMSO control. The average of each triplicate was calculated and the mean of all replicates was plotted. Error bars represent the standard error of means (SEM).

### Chromatin Immunoprecipitation Sequencing (ChIPseq)

Cells were sequentially cross-linked using 2 mM DSG disuccinimidyl glutarate for 30 min and a final concentration of 1% formaldehyde for 10 min at room temperature (20-25° C.) and stopped with 125 mM glycine. Cells were lysed in lysis buffer (20 mM Tris-HCl at pH 7.5. 300 mM NaCl, 2 mM EDTA, 0.5% NP40, 1% Triton X-100, 1 mM PMSF, PIC) and incubated on ice for 30 min. The resuspended cells were then dounced in an ice-cold homogenizer. Nuclear pellets were collected and resuspended in shearing buffer (0.1% SDS, 0.5% N-lauroylsarcosine, 1% Triton X-100, 10 mM Tris-HCl at pH 8.1, 100 mM NaCl, 1 mM EDTA. 1 mM PMSF, PIC). Isolated chromatin was fragmented to an average size of 200-600 bp with a bioruptor (Diagenode). Precleared chromatin was immunoprecipitated overnight at 4° C. and immunocomplexes were collected with protein A Dynabeads. The immunocomplexes were washed eight times in wash buffer (50 mM HEPES-KOH at pH 7.6, 500 mM LiCl, 1 mM EDTA. 1% NP40, 0.7% sodium deoxycholate, 1 mM PMSF, PIC), followed by two 1×TE washes, and eluted in elution buffer (50 mM Tris-HCl at pH 8.0, 10 mM EDTA, 1% SDS), crosslinks were reversed at 65° C. for 4 h or overnight, and DNA was purified using DNA Clean & Concentrator Kit according to the manufacturer's instructions. ChIP- or input-DNA was used for Illumina library construction using ThruPlex DNA-seq kit (Takara) with 12 to 14 cycles of amplification and the use of single indexing barcodes. Paired-end sequencing (37 bp) was performed on a NextSeq500 platform (Illumina). Raw Illumina NextSeq BCL files were converted to FASTQ using Illumina bcl2fastq. Reads were aligned to human GRCh38/hg38 genome using STAR 2.7.5. Aligned BAM files were sorted, duplicate reads marked and removed, and deduplicated BAMs indexed using Broad picard tools v2.9.4. Signal intensities around transcription start sites (TSS, +/−3000 bp) were quantified using BEDtools (v. 2.28.0) and data visualizations were produced using IGVtools (TDF signal pileups; v2.3.75) and ngs.plot (pileup heatmaps/torpedo plots). ChIPseq peaks were called using MACS2 (v. 2.1.4.) with appropriate input samples used as controls.

### RNA Sequencing (RNAseq)

Total RNA was isolated from cell lines or mouse cell depleted PDX material using the RNeasy™ Mini Kit (QIAGEN). Quality control was performed using RNA-Tape™ (Agilent) and all samples used for sequencing passed QC with a RINe-score greater than 8. Poly(A) mRNA enrichment and library preparation was performed using the NEBNext Poly(A) mRNA Magnetic Isolation Module and NEBNext Ultra II RNA Library Prep kit (New England BioLabs) according to the manufacturer's instructions. Sequencing was done on an Illumina NextSeq500 platform as 37 bp paired end sequencing. Raw Illumina NextSeq BCL files were converted to FASTQ using Illumina bcl2fastq. Reads were aligned to human GRCh38/hg38 genome using STAR 2.7.5. Aligned BAM files were sorted, duplicate reads marked and removed, and deduplicated BAMs indexed using Broad picard tools v2.9.4. Raw per-gene counts were calculated with HTSeq-count (v.0.6.1pl). Differential RNA-seq expression was calculated using the BioConductor DESeq2 package (v1.24,0), using raw unnormalized per-gene counts from deduplicated BAMs. Z-scores for the generation of heatmaps were calculated from regularized gene-counts (from DeSeq2 package).

### Statistical Analysis

Statistics were performed using GraphPad Prism software (GraphPad Software, San Diego, CA). Statistical analysis of Menin inhibitor dose-response differences between MEN1-WT and MEN1-mutant cell lines was performed using unpaired Student's t-test (two-tailed) (FIG. 3, FIG. 9, and FIG. 10). The assessment of statistical differences in Menin and MLL1 TSS-occupancy from ChIPseq data was performed using Mann-Whitney-U-test (two-tailed), since this data is not normally distributed (FIG. 4 and FIG. 11). Statistical comparison of disease burden and induction of differentiation in PDX across different conditions was performed using one-way ANOVA (FIG. 6 and FIG. 7). P<0.05 was considered statistically significant. Legend for stars used to indicate significance in dose-response curves (FIG. 3, FIG. 9, and FIG. 10): * p<0.05, ** p<0.01, *** p<0.001.

### Data Availability

All raw and processed sequencing data are accessible via the NCBI Gene-Expression Omnibus (GEO) under the accession number: GSE196037 (ChIPseq: GSE196036; RNAseq: GSE196035). X-ray crystal structures will be made publicly available via the Protein Data Bank (PDB). Scripts for structure preparation, docking, and simulation can be found on Github (github.com/choderalab/men1). All storage intensive files (ie. MSM structures, transition matrices, weights, strided trajectories, etc.) can be found on OSF (osf.io/uge5j/). The complete dataset of trajectories (450 GB total storage needed) is available upon request.

### REFERENCES

1 Rodrigues, C. P., Shvedunova, M. & Akhtar, A. Epigenetic Regulators as the Gatekeepers of Hematopoiesis. Trends Genet (2020). doi.org:10.1016/j.tig.2020.09.015
2 Uckelmann, H. J. & Armstrong, S. A. Chromatin Complexes Maintain Self-Renewal of Myeloid Progenitors in AML: Opportunities for Therapeutic Intervention. Stem cell reports 15, 6-12 (2020). doi.org:10.1016/j.stemcr.2020.05.013
3 Kuhn, M. W. et al. Targeting Chromatin Regulators Inhibits Leukemogenic Gene Expression in NPM1 Mutant Leukemia. Cancer discovery 6, 1166-1181 (2016). doi.org:10.1158/2159-8290.CD-16-0237
4 Yokoyama. A. & Cleary. M. L. Menin critically links MLL proteins with LEDGF on cancer-associated target genes. Cancer cell 14, 36-46 (2008). doi.org:10.1016/j.ccr.2008.05.003
5 Yokoyama. A. et al. The menin tumor suppressor protein is an essential oncogenic cofactor for MLL-associated leukemogenesis. Cell 123, 207-218 (2005). doi.org:10.1016/j.cell.2005.09.025
6 Stein, E. M. et al. in American Society of Hematology Annual Meeting & Exposition 2021 (Atlanta, GA, 2021).
7 Huang, J. et al. The same pocket in menin binds both MLL and JUND but has opposite effects on transcription. Nature 482, 542-546 (2012). doi.org:10.1038/nature10806
8 Hughes, C. M. et al. Menin associates with a trithorax family histone methyltransferase complex and with the hoxc8 locus. Molecular cell 13, 587-597 (2004). doi.org:10.1016/s1097-2765(04)00081-4
9 Klossowski, S. et al. Menin inhibitor MI-3454 induces remission in MLL1-rearranged and NPM1-mutated models of leukemia. The Journal of clinical investigation 130, 981-997 (2020). doi.org:10.1172/JCI129126
10 Lei, H. et al. Recent Progress of Small Molecule Menin-MLL Interaction Inhibitors as Therapeutic Agents for Acute Leukemia. Journal of medicinal chemistry 64, 15519-15533 (2021). doi.org:10.1021/acs.jmedchem.1c00872
11 Perner, F. & Armstrong, S. A. Targeting Chromatin Complexes in Myeloid Malignancies and Beyond: From Basic Mechanisms to Clinical Innovation. Cells 9 (2020). /doi.org:10.3390/cells9122721
12 Uckelmann, H. J. et al. Therapeutic targeting of preleukemia cells in a mouse model of NPM1 mutant acute myeloid leukemia. Science 367, 586-590 (2020). doi.org:10.1126/science.aax5863
13 Krivtsov, A. V. et al. A Menin-MLL Inhibitor Induces Specific Chromatin Changes and Eradicates Disease in Models of MLL-Rearranged Leukemia. Cancer cell 36, 660-673 e611 (2019). doi.org:10.1016/j.ccell.2019.11.001
14 Lemos, M. C. & Thakker, R. V. Multiple endocrine neoplasia type 1 (MEN1): analysis of 1336 mutations reported in the first decade following identification of the gene. Hum Mutat 29, 22-32 (2008). doi.org: 10.1002/humu.20605
15 Hanna, R. E. et al. Massively parallel assessment of human variants with base editor screens. Cell 184, 1064-1080 e1020 (2021). doi.org:10.1016/j.cell.2021.01.012
16 Zimmerman, M. I. et al. SARS-CoV-2 simulations go exascale to predict dramatic spike opening and cryptic pockets across the proteome. Nature Chemistry 13, 651-659 (2021). doi.org:10.1038/s41557-021-00707-0
17 Ward, M. D. et al. Deep learning the structural determinants of protein biochemical properties by comparing structural ensembles with DiffNets. Nature Communications 12. 3023 (2021). doi.org:10.1038/s41467-021-23246-1
18 Sun, X., Singh, S., Blumer, K. J. & Bowman, G. R. Simulation of spontaneous G protein activation reveals a new intermediate driving GDP unbinding. eLife 7 (2018). doi.org:10.7554/eLife.38465
19 Grembecka, J., Belcher, A. M., Hartley, T. & Cierpicki, T. Molecular basis of the mixed lineage leukemia-menin interaction: implications for targeting mixed lineage leukemias. The Journal of biological chemistry 285, 40690-40698 (2010). doi.org:10.1074/jbc.M110.172783
20 Bai, H. et al. Menin-MLL protein-protein interaction inhibitors: a patent review (2014-2021). Expert Opin Ther Pat 32, 507-522 (2022). doi.org:10.1080/13543776.2022.2045947
21 Ross, D. S. et al. Immunohistochemical analysis of estrogen receptor in breast cancer with ESRI mutations detected by hybrid capture-based next-generation sequencing. Modern pathology: an official journal of the United States and Canadian Academy of Pathology, Inc 32, 81-87 (2019). doi.org:10.1038/s41379-018-0116-5
22 Biancaniello, C. et al. Investigating the Effects of Amino Acid Variations in Human Menin. Molecules 27 (2022). doi.org:10.3390/molecules27051747
23 Nikolovska-Coleska, Z. et al. Development and optimization of a binding assay for the XIAP BIR3 domain using fluorescence polarization. Anal Biochem 332, 261-273 (2004). doi.org:10.1016/j.ab.2004.05.055

### Example 3

Acute leukemias harboring rearrangements of the Lysine Methyltransferase 2A (KMT2Ar) gene have a very poor prognosis, and mutations in the Nucleophosmin 1 (NPM1) gene represent the most common genetic abnormality found in acutemyeloid leukemia (AML), occurring in up to 30% of adult patients. Both KMT2Ar and NPM1-mutant acute leukemias are driven by aberrant expression of HOX genes, which co-opt a gene expression program that transforms cells. Menin, a chromatin adaptor protein, is needed for the formation of conserved chromatin complexes and is required for the development of HOX gene-overexpressing acute leukemias driven by KMT2Ar and NPM1 mutations.

Inhibitors designed to disrupt the Menin-MLL1 interaction demonstrate potent disease-eradicating activity in pre-clinical models. The Menin inhibitor SNDX-5613 has been reported to be safe and effective in patients with relapsed/refractory acute leukemia with KMT2Ar or an NPM1c mutation. The phase 1/2 first-in-human study (AUGMENT-101) of SNDX-5613 in this patient population demonstrated an overall response rate of 53%. Here, we identified somatic mutations within the MEN1 gene that arise during Menin inhibitor treatment and mediate therapeutic resistance.

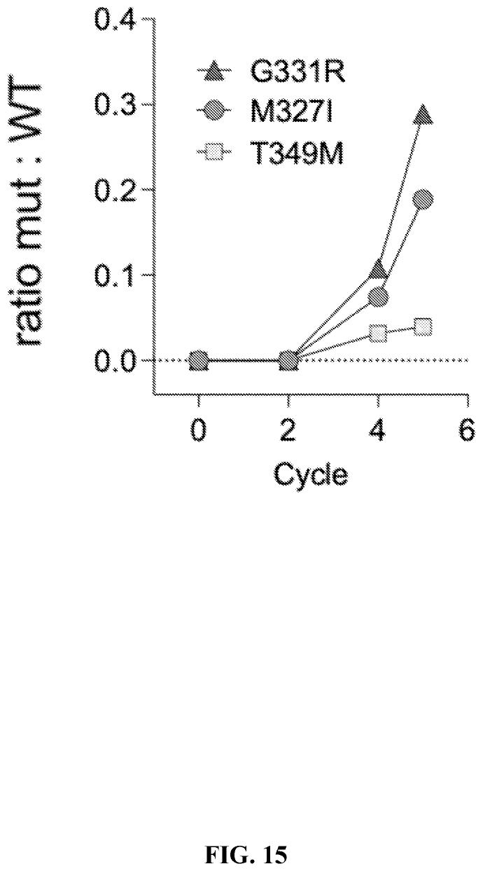

We identified patients who developed resistance to SNDX-5613 after an initial response. Next-generation sequencing of bone marrow specimens at time of loss of response revealed somatic mutations in the MEN1 gene at residues M327, T349, G331, and S160. These mutations have never been described and are distinct from known MEN1 variants that disrupt its tumor suppressive function in MEN1 syndrome. To assess the frequency of these somatic mutations within the population of KMT2Ar and NPM1c-mutant patients exposed to SNDX-5613, we performed droplet digital PCR (ddPCR) for MEN1 mutations on samples collected during the multi-center AUGMENT-101 trial (FIG. 15). We identified 12 of 31 patients (38.7%) who were treated for >2 cycles (>56 days) and acquired one or more MEN1 mutations. These mutations were not detected in the pre-treatment samples by ddPCR (FIG. 15), indicating that they were either acquired on treatment or in very low abundance at baseline.

We also conducted a MEN1-focused CRISPR-Cas9 base-editor screen in KMT2Ar human cell lines exposed to vehicle or Menin inhibitor. This screen validated residues T349, G331, and S160 as determinants of Menin inhibitor resistance. We also treated 5 different patient-derived xenografts (PDX) derived from AML patients with Menin inhibitor. MEN1 mutations were identified in 15 out of 22 (68%) of the relapsed PDX models. MEN1-M327V, -M327I, -G331R, -G331D, -T349M and -S160C mutations were detected at relapse, establishing high concordance with the mutated residues found in patients.

NPM1-mutant and KMT2Ar human cell lines harboring resistance mutations were derived via lentiviral expression or CRISPR-editing. Expression of MEN1-M327I, -G331R and -T349M mutants was sufficient to establish resistance to SNDX-5613 as assessed by cell growth assays and myeloid differentiation. This resistance phenotype could similarly be observed when cells were treated with several Menin inhibitor chemotypes, including MI-3454, indicating that these mutations mediate resistance across the classes of currently available Menin inhibitors. ChIP-seq for MLL1/Menin occupancy on chromatin, coupled with RNA-seq, demonstrated that restoration of HOX gene expression was associated with a defect in the ability of SNDX-5613 to evict mutant Menin/MLL1 from chromatin.

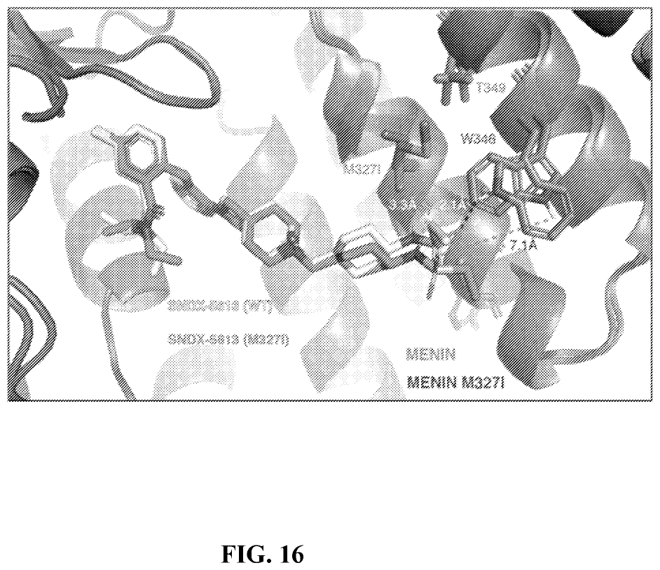

Structural and biochemical studies provided mechanistic insights into how these mutated residues restored Menin/MLL1 chromatin complexes. Fluorescence polarization and isothermal calorimetry assays demonstrated over 50-fold reductions in affinity between SNDX-5613 and MEN1-M327I when compared to MEN1-WT. Furthermore, X-ray co-crystal structures of SNDX-5613 bound to wild-type or mutant Menin showed how perturbation of residue M327 leads to disruption of H-bonds with W346 that uniquely affects inhibitor binding without disrupting the Menin-MLL1 interaction (FIG. 16). Taken together, this study demonstrates for the first time that a chromatin-targeting drug elicits genetic escape mutants that allow for retention of chromatin complexes to sustain a leukemogenic gene expression program as a mechanism of therapeutic resistance.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

1. A method for diagnosing resistance to a menin-inhibitory therapeutic in a subject having cancer or suspected of having cancer, comprising detecting a mutation in a MEN1 gene in the subject.

2. The method of claim 1, wherein the mutation is detected in a cell from the cancer.

3. The method of claim 2, wherein the cancer comprises an acute leukemia.

4. The method of claim 3, wherein the acute leukemia comprises a myeloid or lymphoblastic acute leukemia.

5. The method of claim 3, wherein the acute leukemia comprises acute myeloid leukemia (AML).

6. The method of claim 2, wherein cells of the cancer have a rearrangement of a Mixed Lineage Leukemia gene (MLLr)(also called KMT2A) or a Nucleophosmin gene (NPM1c).

7. The method of claim 6, wherein a protein encoded by the MMLr contributes to a malignant phenotype of the cancer in presence of menin.

8. The method of claim 2, wherein a malignant phenotype of the cancer is dependent on menin protein.

9. The method of claim 1, wherein menin-inhibitory therapeutics block or decrease interaction of menin protein with a MLL1/MLLr protein and/or decrease drug-induced displacement of a menin complex from chromatin.

10. The method of claim 1, wherein the menin-inhibitory therapeutic comprises SNDX-5613, VTP-50469, MI-3454 or MI-89.

11. The method of claim 1, wherein the menin-inhibitory therapeutic comprises MCP-1, ML227, ML399, MIV-6, M-525, M-89, M-808, MI-2, MI-3, MI-2-2, MI-136, MI-463, MI-505, MI-538, BAY-155, MI-1481 or MI-3454.

12. The method of claim 1, wherein the menin-inhibitory therapeutic comprises Ziftomenib (KO-539), JNJ-75276617, DSP-5336, DS-1594b or BMF-219.

13. The method of claim 1, wherein the subject has been treated with a menin-inhibitory therapeutic.

14. The method of claim 13, wherein the subject is relapsed or is not responsive to the menin-inhibitory therapeutic.

15. The method of claim 1, wherein the mutation in the MEN1 gene substitutes an amino acid for a wild-type amino acid in menin protein at positions 327, 331, 349, 160, or combinations thereof.

16. The method of claim 15, wherein the mutation in the MEN1 gene substitutes an amino acid for a wild-type amino acid in menin protein at one, two, three or all of positions 327, 331, 349 and 160.

17. The method of claim 15, wherein the wild-type amino acid at position 327 comprises methionine, at position 331 comprises glycine, at position 349 comprises threonine, at 160 comprises serine, or combinations thereof.

18. The method of claim 15, wherein the wild-type amino acid is substituted with an isoleucine, valine, arginine, aspartic acid, methionine, cysteine or combinations thereof.

19. The method of claim 15, wherein the wild-type amino acid is substituted with an isoleucine or valine at position 327, an arginine or aspartic acid at position 331, a methionine at position 349 and/or a cysteine at position 160.

20. The method of claim 17, wherein the mutation in the MEN1 gene substitutes the wild-type methionine with isoleucine or valine at position 327.

21. The method of claim 17, wherein the mutation in the MEN1 gene substitutes the wild-type glycine with arginine or aspartic acid at position 331.

22. The method of claim 17, wherein the mutation in the MEN1 gene substitutes the wild-type threonine with methionine at position 349.

23. The method of claim 17, wherein the mutation in the MEN1 gene substitutes the wild-type serine with cysteine at position 160.

24. The method of claim 17, wherein the mutation in the MEN1 gene substitutes the wild-type methionine with isoleucine or valine at position 327 and substitutes the wild-type glycine with arginine at position 331.

25. The method of claim 17, wherein the mutation in the MEN1 gene substitutes the wild-type methionine with isoleucine at position 327 and substitutes the wild-type glycine with arginine at position 331.

26. The method of claim 15, wherein substitution of the wild-type amino acid at position 349 occurs in cancers that have substitutions of the wild-type amino acid at positions 327, 331, or both 327 and 331.

27. The method of claim 15, wherein substitution of the wild-type amino acids at positions 327, 331, 349 and 160 are all found in cells of a cancer in the subject.

28. The method of claim 15, wherein substitution of the wild-type amino acids at positions 327, 331, 349 and 160 are all found in single cells of a cancer in the subject.

29. A method for diagnosing resistance of a cancer to a menin inhibitor, comprising:
obtaining a sample comprising cancer cells from a patient; and
testing the cancer cells for presence of a mutation in a MEN1 gene in the patient.

30. The method of claim 29, wherein the sample comprises blood, bone marrow or spinal fluid.

31. The method of claim 29, wherein the testing uses polymerase chain reaction (PCR) or nucleotide sequencing.

32. A method for treating a cancer patient who is receiving a menin-inhibitory therapeutic, comprising:
performing the method of claim 1 on cancer cells obtained from the patient; and
discontinuing treatment with the menin-inhibitory therapeutic if a mutation in a MEN1 gene from the patient is detected.
